SEQUENCE LISTING (21 sequences)

SEQ ID NO: 1 - DNA, 954 bp, Artificial Sequence
  Open reading frame encoding SEQ ID NO2 conveying enhanced protein expression
atgggcgtcgatccgtttgaacgtaacaaaatcttgggccgcggcattaatatcggcaatgcgctcgaagcaccaaatgaaggcgactggggagtggtgataaaagatgagttcttcgacattataaaagaagccggtttctctcatgttcgaattccaataagatggagtacgcacgctcaggcgtttcctccttataaaatcgagccttctttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctttaataggaggagatagcattgaatga

SEQ ID NO: 2 - protein, 317 aa, Thermotoga maritima
MGVDPFERNKILGRGINIGNALEAPNEGDWGVVIKDEFFDIIKEAGFSHVRIPIRWSTHAQAFPPYKIEPSFFKRVDEVINGALKRGLAVVINIHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHLIEEFNFIEEWSKKNKRPIYIGEFGAYRKADLESRIKWTSFVVREAEKRGWSWAYWEFCSGFGVYDPLRKQWNKDLLEALIGGDSIE

SEQ ID NO: 3 - DNA, 954 bp, Artificial Sequence
  Open reading frame encoding SEQ ID NO2
atgggtgttgatccttttgaaaggaacaaaatattgggaagaggcattaatataggaaatgcgcttgaagcaccaaatgagggagactggggagtggtgataaaagatgagttcttcgacattataaaagaagccggtttctctcatgttcgaattccaataagatggagtacgcacgctcaggcgtttcctccttataaaatcgagccttctttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctttaataggaggagatagcattgaataa

SEQ ID NO: 4 - DNA, 976 bp, Artificial Sequence
  5′ region and open reading frame encoding SEQ ID NO2 having modest increased expression
tctactagttaggaggtaacttatgggcgtcgatccgtttgaacgtaacaaaatcttgggccgcggcattaatatcggcaatgcgctcgaagcaccaaatgaaggcgactggggagtggtgataaaagatgagttcttcgacattataaaagaagccggtttctctcatgttcgaattccaataagatggagtacgcacgctcaggcgtttcctccttataaaatcgagccttctttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctttaataggaggagatagcattgaatga

SEQ ID NO: 5 - DNA, 954 bp, Thermotoga maritima
atgggtgttgatccttttgaaaggaacaaaatattgggaagaggcattaatataggaaatgcgcttgaagcaccaaatgagggagactggggagtggtgataaaagatgagttcttcgacattataaaagaagccggtttctctcatgttcgaattccaataagatggagtacgcacgcttacgcgtttcctccttataaaatcatggatcgcttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactataattataggcacagctgaatgggggggtatatctgcccttgaaaaactgtctgtcccaaaatgggaaaaaaattctatagttacaattcactactacaatcctttcgaatttacccatcaaggagctgagtgggtggaaggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaaatggagaaaaggagatggagctgggcatactgggaattttgttccggttttggtgtttatgatactctgagaaaaacctggaataaagatcttttagaagctttaataggaggagatagcattgaataa

SEQ ID NO: 6 - protein, 317 aa, Thermotoga maritima [sequence omitted]

SEQ ID NO: 7 - DNA, 954 bp, Artificial Sequence
  Synthetically generated polynucleotide
atgggtgttgatccttttgaaaggaacaaaatattgggaagaggcattaatataggaaatgcgcttgaagcaccaaatgagggcgactggggagtcgtgataaaagatgagttcttcgacattataaaagaagccggtttctctcatgttcgaattccaataagatggagtacgcacgcttacgcgtttcctccttataaaatcatggatcgcttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactataattataggcacagctgaatgggggggtatatctgcccttgaaaaactgtctgtcccaaaatgggaaaaaaattctatagttacaattcactactacaatcctttcgaatttacccatcaaggagctgagtgggtggaaggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaaatggagaaaaggagatggagctgggcatactgggaattttgttccggttttggtgtttatgatactctgagaaaaacctggaataaagatcttttagaagctttaataggaggagatagcattgaataa

SEQ ID NO: 8 - DNA, 974 bp, Artificial Sequence
  Synthetically generated polynucleotide
atgggtgttgatccttttgaaaggaacaaaatattgggaagaggcattaatataggaaatgcgcttgaagcaccaaatgagggagactggggagtggtgataaaagatgagtatttcgacattataaaagaagccggtttctctcatgttcgaattccaataagatggagtacgcacgctcaggcgtttcctccttataaaatcgaggatcgcttcttcaaaagagtggatgaagtgataaacggagccctgaaaagaggactggctgttgttataaatcagcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactataattataggcacagctgaatgggggggtatatctgcccttgaaaaactgagggtcccaaaatgggaaaaaaatgcgatagttacaattcactactacaatcctttcgaatttacccatcaaggagctgagtgggtggaaggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagctgagaaaaggagatggagctgggcatactgggaattttgttccggttttggtgtttatgatactctgagaaaaacctggaataaagatcttttagaagctttaataggaggagatagcattgaataacaccattccaagatggcgtg

SEQ ID NO: 9 - protein, 317 aa, Artificial Sequence
  Synthetically generated polypeptide
MGVDPFERNKILGRGINIGNALEAPNEGDWGVVIKDEYFDIIKEAGFSHVRIPIRWSTHAQAFPPYKIEDRFFKRVDEVINGALKRGLAVVINQHHYEELMNDPEEHKERFLALWKQIADRYKDYPETLFFEILNEPHGNLTPEKWNELLEEALKVIRSIDKKHTIIIGTAEWGGISALEKLRVPKWEKNAIVTIHYYNPFEFTHQGAEWVEGSEKWLGRKWGSPDDQKHLIEEFNFIEEWSKKNKRPIYIGEFGAYRKADLESRIKWTSFVVREAEKRRWSWAYWEFCSGFGVYDTLRKTWNKDLLEALIGGDSIE

SEQ ID NO: 10 - DNA, 954 bp, Artificial Sequence
  Synthetically generated polynucleotide
atgggtgttgatccttttgaaaggaacaaaatattgggaagaggcattaatataggaaatgcgcttgaagcaccaaatgagggagactggggagtggtgataaaagatgagtatttcgacattataaaagaagccggtttctctcatgttcgaattccaataagatggagtacgcacgctcaggcgtttcctccttataaaatcgaggattctttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatgtgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctctaataggaggagatagcattgaataa

SEQ ID NO: 11 - protein, 317 aa, Artificial Sequence
  Synthetically generated polypeptide
MGVDPFERNKILGRGINIGNALEAPNEGDWGVVIKDEYFDIIKEAGFSHVRIPIRWSTHAQAFPPYKIEDSFFKRVDEVINGALKRGLAVVINIHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHVIEEFNFIEEWSKKNKRPIYIGEFGAYRKADLESRIKWTSFVVREAEKRGWSWAYWEFCSGFGVYDPLRKQWNKDLLEALIGGDSIE

SEQ ID NO: 12 - DNA, 954 bp, Artificial Sequence
  Synthetically generated polynucleotide
atgggtgttgatccttttgaaaggaacaaaatattgggaagaggcattaatataggaaatgcgcttgaagcaccaaatgagggagactggggagtggtgataaaagatgagttcttcgacattataaaagaagccggtttctctcatgttcgaattccaataagatggagtacgcacgctcaggcgtttcctccttataaaatcgaggattctttcttcaaaagagtggatgaagtgataaacggagccctgaaaagaggactggctgttgttataaatcagcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctttaataggaggagatagcattgaataa

SEQ ID NO: 13 - protein, 317 aa, Artificial Sequence
  Synthetically generated polypeptide
MGVDPFERNKILGRGINIGNALEAPNEGDWGVVIKDEFFDIIKEAGFSHVRIPIRWSTHAQAFPPYKIEDSFFKRVDEVINGALKRGLAVVINQHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHLIEEFNFIEEWSKKNKRPIYIGEFGAYRKADLESRIKWTSFVVREAEKRGWSWAYWEFCSGFGVYDPLRKQWNKDLLEALIGGDSIE

SEQ ID NO: 14 - DNA, 972 bp, Thermotoga sp.
atggaacagtcagttgctgaaagtgatagcaactcagcatttgaatacaacaaaatggtaggtaaaggagtaaatattggaaatgctttagaagctcctttcgaaggagcttggggagtaagaattgaggatgaatattttgagataataaagaaaaggggatttgattctgttaggattcccataagatggtcagcacatatatccgaaaagccaccatatgatattgacaggaatttcctcgaaagagttaaccatgttgtcgatagggctcttgagaataatttaacagtaatcatcaatacgcaccattttgaagaactctatcaagaaccggataaatacggcgatgttttggtggaaatttggagacagattgcaaaattctttaaagattacccggaaaatctgttctttgaaatctacaacgagcctgctcagaacttgacagctgaaaaatggaacgcactttatccaaaagtgctcaaagttatcagggagagcaatccaacccggattgtcattatcgatgctccaaactgggcacactatagcgcagtgagaagtctaaaattagtcaacgacaaacgcatcattgtttccttccattactacgaacctttcaaattcacacatcagggtgccgaatgggttaatcccatcccacctgttagggttaagtggaatggcgaggaatgggaaattaaccaaatcagaagtcatttcaaatacgtgagtgactgggcaaagcaaaataacgtaccaatctttcttggtgaattcggtgcttattcaaaagcagacatggactcaagggttaagtggaccgaaagtgtgagaaaaatggcggaagaatttggattttcatacgcgtattgggaattttgtgcaggatttggcatatacgatagatggtctcaaaactggatcgaaccattggcaacagctgtggttggcacaggcaaagagtaa

SEQ ID NO: 15 - protein, 323 aa, Thermotoga sp.
MEQSVAESDSNSAFEYNKMVGKGVNIGNALEAPFEGAWGVRIEDEYFEIIKKRGFDSVRIPIRWSAHISEKPPYDIDRNFLERVNHVVDRALENNLTVIINTHHFEELYQEPDKYGDVLVEIWRQIAKFFKDYPENLFFEIYNEPAQNLTAEKWNALYPKVLKVIRESNPTRIVIIDAPNWAHYSAVRSLKLVNDKRIIVSFHYYEPFKFTHQGAEWVNPIPPVRVKWNGEEWEINQIRSHFKYVSDWAKQNNVPIFLGEFGAYSKADMDSRVKWTESVRKMAEEFGFSYAYWEFCAGFGIYDRWSQNWIEPLATAVVGTGKE

SEQ ID NO: 16 - DNA, 1042 bp, Artificial Sequence
  Synthetically generated polynucleotide
ggatccaccatgagggtgttgctcgttgccctcgctctcctggctctcgctgcgagcgccaccagcggcgtggacccgttcgagaggaacaagatcctgggcaggggcatcaacatcggcaacgccctggaggccccgaacgagggcgactggggcgtggtgatcaaggacgagtacttcgacatcatcaaggaggccggcttcagccacgtgagaatcccgatcaggtggagcacccacgcccaggccttcccgccgtacaagatcgaggacaggttcttcaagagggtggacgaggtgatcaacggcgccctgaagaggggcctggccgtggtgatcaaccagcaccactacgaggagctgatgaacgacccggaggagcacaaggagaggttcctggccctgtggaagcagatcgccgacaggtacaaggactacccggagaccctgttcttcgagatcctgaacgagccgcacggcaacctgaccccggagaagtggaacgagctgctggaggaggccctgaaggtgatcaggagcatcgacaagaagcacaccatcatcatcggcaccgccgagtggggcggcatcagcgccctggagaagctgagggtgccgaagtgggagaagaacgccatcgtgaccatccactactacaacccgttcgagttcacccaccagggcgccgagtgggtggagggcagcgagaagtggctgggcaggaagtggggcagcccggacgaccagaagcacctgatcgaggagttcaacttcatcgaggagtggagcaagaagaacaagaggccgatctacatcggcgagttcggcgcctacaggaaggccgacctggagagcaggatcaagtggaccagcttcgtggtgagggaggccgagaagaggaggtggagctgggcctactgggagttctgcagcggcttcggcgtgtacgacaccctgaggaagacctggaacaaggacctgctggaggccctgatcggcggcgacagcatcgagagcgagaaggacgagctgtgagagctca

SEQ ID NO: 17 - protein, 341 aa, Artificial Sequence
  Synthetically generated polypeptide
MRVLLVALALLALAASATSGVDPFERNKILGRGINIGNALEAPNEGDWGVVIKDEYFDIIKEAGFSHVRIPIRWSTHAQAFPPYKIEDRFFKRVDEVINGALKRGLAVVINQHHYEELMNDPEEHKERFLALWKQIADRYKDYPETLFFEILNEPHGNLTPEKWNELLEEALKVIRSIDKKHTIIIGTAEWGGISALEKLRVPKWEKNAIVTIHYYNPFEFTHQGAEWVEGSEKWLGRKWGSPDDQKHLIEEFNFIEEWSKKNKRPIYIGEFGAYRKADLESRIKWTSFVVREAEKRRWSWAYWEFCSGFGVYDTLRKTWNKDLLEALIGGDSIESEKDEL

SEQ ID NO: 18 - DNA, 1042 bp, Artificial Sequence
  Synthetically generated polynucleotide
ggatccaccatgagggtgttgctcgttgccctcgctctcctggctctcgctgcgagcgccaccagcggcgtggacccgttcgagaggaacaagatcctgggcaggggcatcaacatcggcaacgccctggaggccccgaacgagggcgactggggcgtggtgatcaaggacgagttcttcgacatcatcaaggaggccggcttcagccacgtgagaatcccgatcaggtggagcacccacgcccaggccttcccgccgtacaagatcgagccgagcttcttcaagagggtggacgaggtgatcaacggcgccctgaagaggggcctggccgtggtgatcaacatccaccactacgaggagctgatgaacgacccggaggagcacaaggagaggttcctggccctgtggaagcagatcgccgacaggtacaaggactacccggagaccctgttcttcgagatcctgaacgagccgcacggcaacctgaccccggagaagtggaacgagctgctggaggaggccctgaaggtgatcaggagcatcgacaagaagcacaccgtgatcatcggcaccgccgagtggggcggcatcagcgccctggagaagctgagggtgccgaagtgggagaagaacgccatcgtgaccatccactactacaacccgttcgagttcacccaccagggcgccgagtgggtgccgggcagcgagaagtggctgggcaggaagtggggcagcccggacgaccagaagcacctgatcgaggagttcaacttcatcgaggagtggagcaagaagaacaagaggccgatctacatcggcgagttcggcgcctacaggaaggccgacctggagagcaggatcaagtggaccagcttcgtggtgagggaggccgagaagaggggctggagctgggcctactgggagttctgcagcggcttcggcgtgtacgacccgctgaggaagcagtggaacaaggacctgctggaggccctgatcggcggcgacagcatcgagagcgagaaggacgagctgtgagagctca

SEQ ID NO: 19 - protein, 341 aa, Artificial Sequence
  Synthetically generated polypeptide
MRVLLVALALLALAASATSGVDPFERNKILGRGINIGNALEAPNEGDWGVVIKDEFFDIIKEAGFSHVRIPIRWSTHAQAFPPYKIEPSFFKRVDEVINGALKRGLAVVINIHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHLIEEFNFIEEWSKKNKRPIYIGEFGAYRKADLESRIKWTSFVVREAEKRGWSWAYWEFCSGFGVYDPLRKQWNKDLLEALIGGDSIESEKDEL

SEQ ID NO: 20 - DNA, 1042 bp, Artificial Sequence
  Synthetically generated polynucleotide
ggatccaccatgagggtgttgctcgttgccctcgctctcctggctctcgctgcgagcgccaccagcggcgtggacccgttcgagaggaacaagatcctgggcaggggcatcaacatcggcaacgccctggaggccccgaacgagggcgactggggcgtggtgatcaaggacgagtacttcgacatcatcaaggaggccggcttcagccacgtgagaatcccgatcaggtggagcacccacgcccaggccttcccgccgtacaagatcgaggacagcttcttcaagagggtggacgaggtgatcaacggcgccctgaagaggggcctggccgtggtgatcaacatccaccactacgaggagctgatgaacgacccggaggagcacaaggagaggttcctggccctgtggaagcagatcgccgacaggtacaaggactacccggagaccctgttcttcgagatcctgaacgagccgcacggcaacctgaccccggagaagtggaacgagctgctggaggaggccctgaaggtgatcaggagcatcgacaagaagcacaccgtgatcatcggcaccgccgagtggggcggcatcagcgccctggagaagctgagggtgccgaagtgggagaagaacgccatcgtgaccatccactactacaacccgttcgagttcacccaccagggcgccgagtgggtgccgggcagcgagaagtggctgggcaggaagtggggcagcccggacgaccagaagcacgtgatcgaggagttcaacttcatcgaggagtggagcaagaagaacaagaggccgatctacatcggcgagttcggcgcctacaggaaggccgacctggagagcaggatcaagtggaccagcttcgtggtgagggaggccgagaagaggggctggagctgggcctactgggagttctgcagcggcttcggcgtgtacgacccgctgaggaagcagtggaacaaggacctgctggaggccctgatcggcggcgacagcatcgagagcgagaaggacgagctgtgagagctca

SEQ ID NO: 21 - protein, 341 aa, Artificial Sequence
  Synthetically generated polypeptide
MRVLLVALALLALAASATSGVDPFERNKILGRGINIGNALEAPNEGDWGVVIKDEYFDIIKEAGFSHVRIPIRWSTHAQAFPPYKIEDSFFKRVDEVINGALKRGLAVVINIHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHVIEEFNFIEEWSKKNKRPIYIGEFGAYRKADLESRIKWTSFVVREAEKRGWSWAYWEFCSGFGVYDPLRKQWNKDLLEALIGGDSIESEKDEL

(19) United States
(12) Patent Application Publication
(10) Pub. No.: US 2020/0115609 A1
(43) Pub. Date: Apr. 16, 2020

(54) CONTROLLED BREAK ENZYME FORMULATIONS

(71) Applicant: BASF Enzymes LLC, San Diego, CA (US)

(72) Inventors: Cristina POP, San Diego, CA (US); Adrienne HUSTON DAVENPORT, Solana Beach, CA (US); Yun HAN, San Diego, CA (US); Michael John PRATT, San Diego, CA (US); Kelvin Ning WONG, San Diego, CA (US); Bin ZHANG, San Diego, CA (US); Dongmei REN, San Diego, CA (US)

(21) Appl. No.: 16/725,264

(22) Filed: Dec. 23, 2019

Related U.S. Application Data
(63) Continuation of application No. 15/300,537, filed on Sep. 29, 2016, now abandoned.
(60) Provisional application No. 61/916,366, filed on Dec. 16, 2013.
(60) Provisional application No. 61/878,224, filed on Sep. 16, 2013.

(51) Int. Cl. C09K 8/035; C12N 9/96; C12N 9/42; C09D 133/00; C09D 127/08; C09D 105/04; C09K 8/62; C09K 8/92; C09K 8/70

(52) U.S. Cl. CPC C09K 8/035; C12Y 302/01004; C12N 9/96; C12N 9/2437; C09D 133/00; E21B 43/26; C09D 105/04; C09K 8/62; C09K 8/92; C09K 8/706; C09D 127/08

(57) ABSTRACT

The present disclosure relates to enzyme formulations and methods of using the enzyme formulations to reduce the viscosity of fluids used in hydrocarbon recovery. Some embodiments provide particles for well treatment, where the particles comprise an acidifier carrier and an enzyme co-encapsulated within a shell. The particles can, for example, allow a delayed or controlled release of the enzyme in a high temperature, high pressure environment.

### CROSS-REFERENCE TO RELATED APPLICATIONS

The present application is a continuation application of U.S. patent application Ser. No. 15/300,537, filed on Sep. 29, 2016, which is a National Phase application under 35 U.S.C. § 371 of PCT/US2014/055668, filed on Sep. 15, 2014, which claims priority under 35 U.S.C. § 119(e) to U.S. Provisional Application No. 61/878,224, filed on Sep. 16, 2013, and U.S. Provisional Application No. 61/916,366, filed on Dec. 16, 2013, the contents of which are herein expressly incorporated by reference in their entireties.

### REFERENCE TO SEQUENCE LISTING

This application is being filed electronically via the USPTO EFS-WEB server, as authorized and set forth in MPEP § 502.05 and this electronic filing includes an electronically submitted sequence listing; the entire content of this sequence listing is hereby incorporated by reference into the specification of this application. The sequence listing is identified on the electronically filed ASCII (.txt) text file as follows:

| File Name | Date of Creation | Size |
|---|---|---|
| 160852US04-SequenceListing.txt | Dec. 18, 2019 | 40.4 KB |
| | | (41,450 bytes) |

### BACKGROUND

### Field

The present disclosure relates to enzyme formulations, method of making the enzyme formulations, and methods of using the enzyme formulations in the field of recovery of hydrocarbons from a subterranean formation, for example, to reduce the viscosity of gelled fluids in a controlled manner.

### Description of the Related Art

Hydraulic fracturing is accomplished by injecting a pressurized fluid, commonly referred to as fracturing fluids, into a subterranean formation at pressures capable of forming fractures in the surrounding earth. Gel or hybrid fracturing fluids can contain a solvent, a gelling agent (viscosifier), proppant, and a breaker. The viscosity of the gelling agent (viscosifier) allows suspension of the proppant within the fluid and a reduced tendency of the proppant settling out during delivery into the rock formation.

Fracturing subterranean formations requires coordination between the gelling agent (viscosifier) and the breaker. Breaking the gelled fracturing fluid (i.e., reducing the viscosity of the gelled fracturing fluid) has commonly been accomplished by adding a “breaker,” that is, a viscosity-reducing agent, to the subterranean formation at the time the break is desired. However, known techniques can be unreliable and result in premature breaking of the gelled fracturing fluid before the fracturing process is complete, and/or incomplete breaking of the gelled fracturing fluid. Premature breaking can cause a decrease in the number of fractures, desired size and geometry of the fractures obtained and proper proppant placement, thus decreasing the potential amount of hydrocarbon recovery due to decreased communication and conductivity of the reservoir to the wellbore. In addition, incomplete breaking can cause a decrease in the well conductivity and thus, the amount of hydrocarbon recovery.

Enzymes have been used as effective and environmentally friendly breakers in recovery of hydrocarbons (e.g., recovery oil, natural gas, etc.) from a subterranean formation. However, the applications of enzyme breakers in hydrocarbon recovery have been limited by, for example, loss of enzymatic activity in the alkaline pH environment of the fracturing liquid and/or at downhole conditions. There is a need for chemically and physically protected enzymes to allow effective break of gelled liquids (e.g., fracturing fluid) at downhole conditions.

### SUMMARY

Some embodiments provide a particle for well treatment. In some embodiments, the particle comprises an enzyme-containing core, wherein the enzyme-containing core comprises an acidifying agent and an enzyme; and a shell configured to at least partially encapsulate the enzyme-containing core.

In some embodiments, the shell allows controlled release of the enzyme from the particle. In some embodiments, the acidifying agent is in the form of solid particle and the acidifying agent serves as a carrier for the enzyme. The enzyme can be present on the outer surface of the enzyme-containing core, dispersed within the enzyme-containing core, or both.

In some embodiments, the enzyme-containing core comprises a binding agent. For example, the binding agent can comprise, or be, polyvinylpyrrolidone, polyvinyl alcohol, alginate, polyethylene glycol, wax, xanthan gum, polyvinyl acetate, carrageenans, starch, maltodextrin, hydroxypropyl cellulose, hydroxypropyl methylcellulose, methylcellulose, carboxymethyl cellulose, styrene acrylic dispersions, or any combination thereof. In some embodiments, the enzyme-containing core comprises an inert carrier. For example, the inert carrier can comprise, or be, fibrous and microcrystalline cellulose, sodium sulfate, sodium chloride, dicalcium phosphate, calcium carbonate, diatomaceous earth, zeolite, starch, or any combination thereof. In some embodiments, the enzyme-containing core comprises a stabilizer. For example, the stabilizer can comprise, or be, mannitol, trehalose, sorbitol, xylitol, sucrose, microcrystalline cellulose, starch, sodium chloride, sodium sulfate, ammonium sulfate, or any combination thereof.

In some embodiments, the acidifying agent comprises a mild acidifying inorganic salt. For example, the mild acidifying inorganic salt can be, or comprise, ammonium sulfate, sodium phosphate monobasic, ammonium chloride, sodium sulfate, potassium sulfate, potassium phosphate monobasic, magnesium chloride, ammonium citrate monobasic, ammonium citrate dibasic, ammonium citrate tribasic, ammonium phosphate monobasic, ammonium phosphate dibasic, sodium phosphate dibasic, potassium phosphate dibasic, sodium citrate monobasic, sodium citrate dibasic, potassium citrate monobasic, potassium citrate dibasic, or any combination thereof. In some embodiments, the acidifying agent comprises an organic acid or a salt thereof. For example, the organic acid can be, or comprise, citric acid, oxalic acid, malonic acid, glycolic acid, pyruvic acid, lactic acid, maleic acid, aspartic acid, isocitric acid, or any combination thereof. In some embodiments, the acidifying agent comprises an ester, a lactone, polyester, polylactone, or any combination thereof. For example, the ester can be an ester of an organic acid.

In some embodiments, the acidifying agent comprises polylactic acid, poly(lactic-co-glycolic acid), diphenyl oxalate, polyglycolic acid, poly(ethylene) therephtalates, polycaprolactone, or any combination thereof. In some embodiments, the acidifying agent comprises one or more buffers. For example, at least one of the one or more buffers is or comprises a Tris-HCl buffer, a morpholino-ethanesulphonic acid (MES) buffer, a pyridine, cacodylate buffer, a Bis(2-hydroxyethyl)amino-tris(hydroxymethyl)methane (BIS-TRIS( ) buffer, a piperazine-N,N′-bis(2-ethanesulfonic acid (PIPES) buffer, a 3-(N-morpholino)propanesulfonic acid (MOPS) buffer, a 3-(N-Morpholino)-2-hydroxypropanesulfonic acid (MOPSO) buffer, an ethylene-diamine-tetraacetic acid (EDTA) buffer, a glycine buffer, and any combination thereof.

In some embodiments, the shell comprises a polymer, a homopolymer, a copolymer, or any combination thereof. In some embodiments, the shell comprises a polymer comprising one or more of the monomers selected from the group consisting of methacrylic acid, methacrylic ester, methacrylic amide, methacrylic nitril, acrylic acid, acrylic ester, acrylic amide, acrylic nitril, and vinyl monomers. For example, the vinyl monomers comprise styrene and alpha methyl styrene. In some embodiments, the shell comprises ethylcellulose, acrylic resin, plastics, methacrylate, acrylate, acrylic acetate, polyvinylidene chloride (PVDC), nitrocellulose, polyurethane, wax, polyethylene, polyethylene glycol, polyvinylalcohol, polyester, polylactic acid, polyglycolic acid, copolymers of polylactic and polyglycolic acids, polyvinyl acetate, vinyl acetate acrylic copolymer, alginates, agar, styrene-acrylate copolymer, styrene/n-butyl acrylic copolymer, or any combination thereof.

In some embodiments, at least one of the enzymes is a cellulase, a hemicellulase, a pectinase, a xanthanase, a mannanase, a galactosidase, or an amylase. The enzyme can be a thermostable or thermotolerant enzyme.

In some embodiments, the particle comprises one or more additional coatings outside of or underneath the shell. In some embodiments, at least one of the additional coatings is a polymeric protective coating or a polymeric polishing coating.

In some embodiments, the size of the particle is about 7 mesh to about 60 mesh on the U.S. Sieve Series. 32. In some embodiments, the size of the particle is about 10 mesh to about 20 mesh on the U.S. Sieve Series.

In some embodiments, the shell substantially encapsulates the enzyme-containing core. In some embodiments, the shell encapsulates the entire enzyme-containing core.

In some embodiments, the particle is configured to reduce the pH of a well treatment composition below a threshold pH value at and above which the composition can reheal. In some embodiments, the threshold pH value is 9.5.

Also disclosed herein are well treatment compositions that comprise one or more the particles for well treatment. In some embodiments, the well treatment composition comprises a plurality of the particles. In some embodiments, the well treatment composition comprises a viscosifier and a solvent. In some embodiments, the well treatment composition further comprises a cross-linking agent. In some embodiments, the well treatment composition is configured to reduce the pH of a cross-linked well treatment fluid below a threshold pH value at and above which the fluid can reheal. In some embodiments, the cross-linked well treatment fluid is a fracturing fluid, a gravel packing fluid, a completion fluid, a workover fluid, a drilling fluid, or any combination thereof. In some embodiments, the threshold pH value is 9.5.

Method of treating a subterranean formation is also disclosed. The method, in some embodiments, can comprise contacting the subterranean formation with a well treatment fluid, wherein the well treatment fluid comprises a plurality of particles for well treatment, a viscosifier and a solvent; and allowing the enzyme to reduce the viscosity of the well treatment fluid.

In some embodiments, the enzyme reduces the viscosity of the well treatment fluid by at least one order of magnitude. In some embodiments, the well treatment fluid is a fracturing fluid, a gravel packing fluid, a completion fluid, a workover fluid, or a drilling fluid, or any combination thereof. In some embodiments, the well treatment fluid reaches a complete break in the absence of an additional pH reducing agent. In some embodiments, the viscosifier comprises guar, substituted guar, cellulose, derivatized cellulose, xanthan, starch, polysaccharide, gelatin, polymer, synthetic polymer, or any combination thereof. In some embodiments, the substituted guar is hydroxylethyl guar, hydroxypropyl guar, carboxymethylhydroxyethyl guar, carboxymethylhydroxypropyl guar (CMHPG), or the derivatized cellulose is carboxymethyl cellulose, polyanoinic cellulose, hydroxyethyl cellulose, or any combination thereof. In some embodiments, the solvent is aqueous or organic-based. In some embodiments, the solvent is fresh water, sea water, brine, produced water, water from aquifers, water with water-soluble organic compounds, or any mixture thereof.

Some embodiments provide a method for making particles for well treatment. In some embodiments, the method comprises contacting an enzyme to with a solid acidifying agent to form an enzyme-containing core; and encapsulating the enzyme-containing core with one or more shells to form the particles for well treatment, wherein each of the shells is configured to at least partially encapsulate the enzyme-containing core. In some embodiments, the contacting step comprises attaching the enzyme to the solid acidifying agent by a non-perforated pan coating process, a pan coating process, a fluidized bed coating process, a spray drying process, or any combination thereof. In some embodiments, the contacting step comprises spraying a solution comprising the enzyme onto the solid acidifying agent.

In some embodiments, the method comprises mixing an enzyme and a solid acidifying agent to form a mixture; granulating the mixture to form an enzyme-containing core; and encapsulating the enzyme-containing core with one or more shells to form the particles for well treatment, wherein each of the shells is configured to at least partially encapsulate the enzyme-containing core.

In some embodiments, the method for making particles for well treatment comprises mixing an enzyme and a solid acidifying agent to form a mixture; granulating the mixture to form an enzyme-containing core; and encapsulating the enzyme-containing core with one or more shells to form the particles for well treatment, wherein each of the shells is configured to at least partially encapsulate the enzyme-containing core.

In some embodiments, the method further comprises drying the enzyme-containing core before encapsulating the enzyme-containing core with the shells. In some embodiments, the mixture further comprises a binder, a stabilizer, an inert carrier, or any combination thereof.

In some embodiments, granulating the mixture to form an enzyme-containing core is achieved by a wet granulation process. In some embodiments, the wet granulation process comprises extrusion, centrifugal extrusion, spheronization, batch high shear granulation, continuous high shear mixing, disc granulation, drum granulation, spray drying, fluid bed agglomeration, fluid bed granulation and/or layering, prilling, or any combination thereof. In some embodiments, the fluid bed granulation and/or layering comprises bottom spray, tangential spray, and spouted bed. In some embodiments, the enzyme-containing core is encapsulated by a non-perforated pan coating process, a pan coating process, a fluidized bed coating process, a spray drying process, or any combination thereof.

In some embodiments, the fluidized bed coating process is a bottom spray process, a Wurster process, a top spray process, a tangential spray process, a spouted bed process, a modified fluidized bed coating process, or a continuous fluidized bed coating process, or any combination thereof.

In some embodiments, the shell comprises a polymer, a homopolymer, a copolymer, or any combination thereof. In some embodiments, the shell comprises a polymer comprising one or more of the monomers selected from the group consisting of methacrylic acid, methacrylic ester, methacrylic amide, methacrylic nitril, acrylic acid, acrylic ester, acrylic amide, acrylic nitril, and vinyl monomers. In some embodiments, the vinyl monomers comprise styrene and alpha methyl styrene. In some embodiments, the shell comprises ethylcellulose, acrylic resin, plastics, methacrylate, acrylate, acrylic acetate, polyvinylidene chloride (PVDC), nitrocellulose, polyurethane, wax, polyethylene, polyethylene glycol, polyvinylalcohol, polyester, polylactic acid, polyglycolic acid, copolymers of polylactic and polyglycolic acids, polyvinyl acetate, vinyl acetate acrylic copolymer, alginates, agar, styrene-acrylate copolymer, styrene/n-butyl acrylic copolymer, or any combination thereof.

In some embodiments, the weight gain of solid content upon encapsulating the enzyme-containing core with the one or more shells is about 20% to about 250%. In some embodiments, the weight gain is about 50% to 150%.

In some embodiments, the encapsulating step comprising curing the particles at an elevated temperature to promote formation of at least one of the shells. In some embodiments, the elevated temperature is between about 25° C. to about 80° C. In some embodiments, the elevated temperature is between about 40° C. to about 60° C.

In some embodiments, the one or more shells are successive shells.

### BRIEF DESCRIPTION OF THE DRAWINGS

FIGS. 1A-B depict illustrative embodiments of methods for making particles for well treatment.

FIGS. 2A-D depict illustrative embodiments of a cross-sectional view of a particle for well treatment that is within the scope of the present disclosure (not to scale).

FIG. 3 is a graph showing the break profile of cross-linked guar compositions treated with free enzyme breaker and acidifiers as described in Example 1.

FIG. 4 is a graph showing the break profile of cross-linked guar compositions treated with acidifiers in the presence or absence of free enzyme breaker as described in Example 2.

FIG. 5 is a table showing the break conditions of cross-linked guar treated with ammonium sulfate acidifier and free enzyme breaker as described in Example 3.

FIG. 6 is a table showing the break conditions of cross-linked guar treated with sodium phosphate monobasic acidifier and free enzyme breaker as described in Example 3.

FIG. 7 is a table showing the break conditions of cross-linked guar treated with citric acid acidifier and free enzyme breaker as described in Example 3.

FIG. 8 is a graph showing the break profiles of cross-linked guar compositions treated with a formulated enzyme breakers as described in Example 4.

FIG. 9 is a graph showing the break profiles of cross-linked guar compositions treated with a formulated enzyme breakers as described in Example 5.

FIG. 10 is a graph showing the break profiles of cross-linked guar compositions treated with a formulated enzyme breaker as described in Example 6.

FIG. 11 is a graph showing the break profiles of a cross-linked guar composition treated with a formulated enzyme breaker as described in Example 7.

FIG. 12 is a graph showing the break profiles of a cross-linked guar composition treated with a formulated enzyme breaker as described in Example 8.

FIG. 13 is a graph showing the break profiles of a cross-linked guar composition treated with a formulated enzyme breaker as described in Example 8.

FIG. 14 is a graph showing the breaking profiles of a cross-linked guar composition treated with formulated enzyme breakers of different shelf life as described in Example 9.

FIG. 15 is a graph showing the breaking profiles of a cross-linked guar composition treated with formulated enzyme breakers as described in Example 10.

FIG. 16 is a graph showing the breaking profile of a cross-linked guar composition treated with non-encapsulated cellulase as described in Example 11.

FIG. 17 is a graph showing the breaking profile of a cross-linked guar composition treated with an encapsulated particle as described in Example 12.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

The present disclosure relates to compositions and methods for treating subterranean formations, for example formulated enzyme breakers and methods for breaking viscosified treatment fluids utilized in the treatment of subterranean formations. For example, particles for well treatment and well treatment compositions that comprise the particles for well treatment are disclosed. In some embodiments, the particles can include an enzyme-containing core comprising an acidifying agent and an enzyme, and a shell configured to at least partially encapsulate the enzyme-containing core.

Also disclosed herein are methods for making and using the compositions for treating subterranean formulations.

### Formulated Enzyme Breakers

As disclosed herein, compositions comprising enzymes capable of reducing viscosity of one or more fluids used in hydrocarbon recovery can be formulated to form formulated enzyme breakers so that the enzyme can be protected chemically and/or physically from, for example, unsuitable temperature, pressure, or pH conditions. For example, formulated enzyme breakers disclosed herein can be added to any subterranean treatment fluid known in the art or a combination there of to reduce its viscosity. Suitable examples of subterranean treatment fluids include, but are not limited to, drilling fluids, fracturing fluids, carrier fluids, diverting fluids, gravel packing fluids, completion fluids, workover fluids, and the like in downhole conditions.

The formulated enzyme breaker compositions disclosed herein, in some embodiments, provide controlled breaking of viscosified subterranean treatment fluids. As disclosed herein, breaking a viscosified treatment fluid refers to the reduction of the viscosity of the viscosified subterranean treatment fluid. Viscosified treatment fluids are typically viscosified by crosslinked gels that are often crosslinked through a crosslinking reaction involving a gelling agent and crosslinking agent.

Typically, for a crosslinking agent to effectively cross-link gelled solutions the pH of the subterranean treatment fluid must be adjusted, for example, at high pH. Above 9.0, the borate ion exists and is available to cross-link and cause gelling. At lower pH, the borate is converted to boric acid, which is not ionized.

H₃BO₃+OH⁻ [symbol] B(OH)4⁻ The pKa for boric acid is 9.14.

Generally, successful fracturing occurs when the fracturing fluid is thoroughly dispersed in the subterranean formation and achieves maximum viscosity and pressure which in turn fractures the surrounding earth, and deposits proppant into said fractures. After the fracturing fluid achieves the maximum viscosity, the fracturing fluid can be broken to a less viscous form, preferably reaching a complete break. As used herein, the term “complete break” refers to a viscosity of the flowback less than 10 cP or less as measured with VISCOlab 4000 from Cambridge Viscosity. The breaking of the cross-linked gelled fluid allows the pressure within the subterranean formation to be relieved, and the less viscous or “broken” gelled fluid to be pumped out of the subterranean formation. A complete break of the fracturing fluid may allow the fracturing fluid to be more completely removed from the fractured subterranean formation with less residue which then increases the conductivity of the outflow of hydrocarbon resource (e.g. oil and or gas).

One aspect of decreasing the viscosity of cross-linked gelled fluid is by adjusting the environmental pH to shift the equilibrium and to reverse the cross-link reaction. For example, guar gum cross-linked with borate may be reversed by reducing the environmental pH, preferably below a pH of 9. By reducing the environmental pH, the borate cross-linking reaction is reversed, and the viscosity of the guar gum is reduced, changing the structure of the guar gum from cross-linked (tertiary) to linear. Linear guar has significantly lower viscosity than cross-linked guar. Acidifiers can reduce environmental pH, and therefore modify the equilibrium between cross-linked guar gum and linear guar gum, favoring accumulation of linear guar gum. However, reversal of the cross-linking reaction is not sufficient to decrease the viscosity to acceptable levels required for water thin flow back, i.e., a complete break. To achieve a complete break or water thin flow back of guar gum, the carbohydrate structure (a.k.a the backbone) of the linear guar gum must be broken, for example by enzyme hydrolysis. Reversal of the cross-linking reaction of the guar gum facilitates significantly the access of the enzyme to carbohydrate bonds within the guar gum, by eliminating steric hindrance and increasing diffusion. Linear guar can then be hydrolyzed by the enzyme to water thin solutions, for example after a complete break, and then the water thin solution can be safely and effectively pumped back to the surface.

To achieve the step-wise synchronization of reaching maximum viscosity of the gelled fluid and a complete break of the gelled fluid, it is advantageous, in some embodiments, to have a “controlled breaking.” For example, the breaking of the gelled fluid can be achieved at certain desirable condition(s) (e.g., environmental conditions) and/or within a desirable amount of time.

In some embodiments, it can be advantageous to have the breaker distributed into the fractures with proppant so that fractures laden with proppant can be cleared of viscous gelled fluid.

The formulated enzyme breakers described herein can be used for controlled breaks, and preferably complete breaks, of gelled subterranean treatment fluids under the environmental conditions typically found in subterranean formations during oil and gas discovery operations.

In some embodiments, cellulase enzyme formulated with an acidifier carrier particle, is encapsulated with a coating which delays the activity of the enzyme and acidifier.

The formulated enzyme breakers disclosed herein comprise, in some embodiments, one or more particles for well treatment. In some embodiments, the particle for well treatment comprises an enzyme-containing core, wherein the enzyme-containing core comprises an acidifying agent and an enzyme; and a shell configured to at least partially encapsulate the enzyme-containing core. The formulated enzyme breakers can be used in various hydrocarbon recovery processes, including but not limited to, breaking subterranean treatment fluids (for example, fracturing fluids, drilling fluids, blocking fluids, carrier fluids, diverting fluids, gravel packing fluids, completion fluids, workover fluids, and the like), and degrading filter cakes.

### Enzyme-Containing Core

The enzyme-containing core of the particles for well treatment can, in some embodiments, comprise one or more acidifying agents and one or more enzymes. The acidifying agent(s) can, for example, serve as carriers for the enzyme(s). As used herein, a “carrier” is any particle to which an enzyme may be affixed by any means known in the art. In some embodiments, the enzyme is attached to the particle in the presence of a binder. For example, the enzyme can be attached to a solid acidifying agent in the presence of a binder.

The enzyme can be present on the surface (e.g., the outer surface) of the enzyme-containing core, and/or the enzyme can be dispersed within the enzyme-containing core. The present disclosure is not particularly limited in how the enzyme is dispersed within the enzyme-containing core. In some embodiments, the enzyme is randomly dispersed within the enzyme-containing core. In some embodiments, the enzyme is dispersed in a predetermined pattern within the enzyme-containing core. Various embodiments are disclosed herein, but others will be readily apparent to the skilled artisans and are within the scope of the present disclosure.

Acidifying Agents

As used herein, the terms “acidifying agent” and “acidifier” are used interchangeably, and refer to any substance that can lower the pH of the environment in which it is present. For example, the acidifying agent can be an organic compound, an inorganic compound, or any combination thereof. In some embodiments, the acidifying agent comprises, or is, an organic acid, or a salt or ester thereof. In some embodiments, the acidifying agent comprises, or is, an inorganic acid, or a salt or ester thereof.

In some embodiments, the acidifying agent comprises mild acidifying inorganic salts, organic acids, salts of organic acids, (poly)esters of organic acids, organic buffers, or any combination thereof. Examples of organic buffers include, but are not limited to, Tris-HCl buffers, morpholino-ethanesulphonic acid (IVIES) buffers, pyridine, cacodylate buffers, Bis(2-hydroxyethyl)amino-tris(hydroxymethyl)methane (BIS-TRIS) buffers, piperazine-N,N′-bis(2-ethanesulfonic acid (PIPES) buffers, 3-(N-morpholino)propanesulfonic acid (MOPS) buffers, 3-(N-Morpholino)-2-hydroxypropanesulfonic acid (MOPSO) buffers, ethylene-diamine-tetraacetic acid (EDTA) buffers, glycine buffers, and any combination thereof. Examples of mild acidifying inorganic salts include, but are not limited to, ammonium sulfate, sodium phosphate monobasic, ammonium chloride, ammonium citrate, sodium sulfate, potassium phosphate monobasic, magnesium chloride, ammonium citrate monobasic, ammonium citrate dibasic, ammonium citrate tribasic, sodium phosphate dibasic, potassium phosphate dibasic, sodium citrate monobasic, sodium citrate dibasic, potassium citrate monobasic, potassium citrate dibasic, and any combination thereof. Non-limiting examples of (poly)esters of organic acid include polylactic acid, poly(lactic-co-glycolic acid), polyglycolic acid, poly(ethylene) therephtalates, polycaprolactone, diphenyl oxalate, and any combination thereof. In some embodiments, the organic acid is citric acid, oxalic acid, malonic acid, glycolic acid, pyruvic acid, lactic acid, maleic acid, aspartic acid, isocitric acid, any salt of these organic acids, or any combination thereof. In some embodiments, the acidifying agent comprises or is an ester, a lactone, polyester, polylactone, or any combination thereof. In some embodiments, the acidifying agent comprises or is an ester. Non-limiting examples of polyester include solid biodegradable polyesters (SBPs), such as polybutylene succinate (PBS), poly(butylene succinate-co-butylene terephthalate (PBBT), polybutylene terephalate, polyhydroxybutyrate, and any combination thereof.

The acidifying agent can be in a solid or liquid form. In some embodiments, it is advantageous to have the acidifying agent in a solid form, for example as solid particles. For example, the acidifying agent can be in a powder form (e.g., fine particles) or a granular form.

The amount of acidifying agent in the particle can vary. For example, the amount of the acidifying agent in the particle can be, or about 1%, 2%, 5%, 10%, 20%, 30%, 40%, 50%, 60%, 70%, 75%, 80%, 85%, 90%, 95%, 98%, 99%, or any range between two of these values (including the end points) by weight, based on the total weight of the particle. In some embodiments, the amount of the acidifying agent in the particle can be at least, or at least about 5%, 10%, 20%, 30%, 40%, 50%, 60%, 70%, 75%, 80%, 85%, 90%, or 95% by weight, based on the total weight of the particle. In some embodiments, the amount of and type of acidifying agent can vary based on the initial pH of the well treatment fluid to be used. For example, well treatment fluids with higher initial pH may require more acidifier or a stronger acidifier than well treatment fluids with a lower initial pH.

In some embodiments, the solid acidifier particles serve as carrier particles to which the enzyme may be attached. In some embodiments, the enzyme is attached to the solid acidifier particles using a binding agent. In some embodiments, the binding agent comprises, or is, polyvinylpyrrolidone, polyvinyl alcohol, alginate, polyethylene glycol, wax (e.g., bee wax, and synthetic wax), xanthan gum, polyvinyl acetate, carrageenans, starch, maltodextrin, hydroxypropyl cellulose, hydroxypropyl methylcellulose, methylcellulose, carboxymethyl cellulose, or any combination thereof. In some embodiments, the solid acidifier serves as a carrier for the enzyme. In some embodiments, the binding agent comprises, or is, any one or more of the encapsulating agents disclosed herein. The term “carrier,” as used in this disclosure, includes solid particulate to which an enzyme composition may be affixed. It is advantageous, in some embodiments, the acidifier cannot lessen or damage the activity of the enzyme upon contact with the enzyme.

The alkaline pH of the cross-linked gelled solutions (e.g. pH 9.5) is not ideal for the activity of most enzyme breakers. Without being bound by any particular theory, it is believed that the acidifier present in the formulated enzyme breakers disclosed herein can, in some embodiments, establish a reduced pH environment upon release in which the enzyme can hydrolyze the cross-linked gelled fluid effectively, preferably to a complete break.

Enzymes

As described herein, the enzyme-containing core can comprise one or more enzymes. The enzyme can be, for example, any enzyme capable of degrading polymeric substances, including but not limited to polysaccharides present in filtercakes, fracturing and blocking gel, as well as in other applications/fluids used in the hydrocarbon recovery. For example, the enzyme can be a hydrolase. Non-limiting examples of the enzyme include cellulases, hemicellulases, pectinases, xanthanases, mannanases, galactosidases, glucanases, amylases, amyloglucosidases, invertases, maltases, endoglucanases, cellobiohydrolases, glucosidases, xylanases, xylosidases, arabinofuranosidases, oligomerases, and the like, and any mixtures thereof. The galactosidases can be α-galactosidases, β-galactosidases, or any combination thereof. The glucosidases can be α-glucosidases, β-glucosidases, or any combination thereof. The amylases can be, for example, α-amylases, β-amylases, γ-amylases, or any combination thereof. In some embodiments, the enzyme is a thermostable or thermotolerant enzyme.

In some embodiments, the enzyme is any of the cellulases derived from hyperthermophilic bacteria and/or non-naturally occurring variants thereof described in PCT publication WO 2009/020459 (the entire disclosure of which is incorporated herein by reference). In some embodiments, the enzyme is encoded by a nucleic acid sequence having at least 70%, 80%, 90%, 95%, 96%, 97%, 98%, 99%, 100%, or a range defined by any two of these values, sequence identity to any of the below-listed DNA sequences described in WO 2009/020459. In some embodiments, the enzyme has an amino acid sequence having at least at least 70%, 80%, 90%, 95%, 96%, 97%, 98%, 99%, 100%, or a range defined by any two of these values, sequence identity to any of the below-listed protein sequences described in WO 2009/020459. The DNA and protein sequences include:

WO 2009/020459 SEQ ID NOS: 1, 2 (wild-type ‘parent’ T. maritima cellulase), disclosed herein as SEQ ID NOs: 5 and 6.

WO 2009/020459 SEQ ID NOS: 3 (wild-type DNA, altered to remove alternate starts) disclosed herein as SEQ ID NO: 7.

WO 2009/020459 SEQ ID NOS: 6, 7 (“7X” combined Gene Site Saturation Mutagenesis (“GSSM”) mutations) disclosed herein as SEQ ID NOs: 8 and 9.

WO 2009/020459 SEQ ID NOS: 8, 9 (“12X-6” combined GSSM mutations), disclosed herein as SEQ ID NOs: 3 and 2.

WO 2009/020459 SEQ ID NOS: 10, 11 (“13X-1” combined GSSM mutations) disclosed herein as SEQ ID NOs: 10 and 11.

WO 2009/020459 SEQ ID NOS: 12, 13 (“12X-1” combined GSSM mutations) disclosed herein as SEQ ID NOs: 12 and 13.

WO 2009/020459 SEQ ID NOS: 16, 17 (alternative cellulase breaker from Thermotoga sp.) disclosed herein as SEQ ID NOs: 14 and 15.

WO 2009/020459 SEQ ID NOS: 18, 19 (“7X” codon-optimized version of T. maritima cellulase for maize expression) disclosed herein as SEQ ID NOs: 16 and 17.

WO 2009/020459 SEQ ID NOS: 20, 21 (“12X-6” codon-optimized version of T. maritima cellulase for maize expression) disclosed herein as SEQ ID NOs: 18 and 19.

WO 2009/020459 SEQ ID NOS: 22, 23 (“13X-1” codon-optimized version of T. maritima cellulase for maize expression) disclosed herein as SEQ ID NOs: 20 and 21.

Besides the above-listed nucleotide and amino acid sequences related to wild-type and evolved variants of the cellulase from Thermotoga maritima strain MSB8, the additional mutants listed in Table 2 and Example 5 (from WO 2009/020459) are also deemed useful as components of the compositions described herein and/or in the methods of making these compositions.

In some embodiments, the enzyme can be a cellulase or a variant of a cellulase disclosed in U.S. Pat. Nos. 5,962,258, 6,008,032, 6,245,547, 7,807,433, international patent publication WO 2009/020459, international patent publication WO 2013/148163, or international patent publication WO 2013/148167, the contents of which are incorporated by reference in their entireties. In some embodiments, the cellulase can be a commercially available product including, but not limited to, PYROLASE® 160 cellulase, PYROLASE® 200 cellulase, or PYROLASE® HT cellulase (Verenium Corp., San Diego, Calif.), or any mixture thereof. In some embodiments, the cellulase is PYROLASE® HT cellulase.

In some embodiments, the enzyme is encoded by a nucleotide sequence set forth in SEQ ID NO: 1, SEQ ID NO: 3, SEQ ID NO:4, SEQ ID NO:5, SEQ ID NO:7, SEQ ID NO:8, SEQ ID NO:10, SEQ ID NO:12, SEQ ID NO:14, SEQ ID NO:16, SEQ ID NO:18, or SEQ ID NO:20. In some embodiments, the enzyme is encoded by a nucleotide sequence that is homologous to the sequence set forth in SEQ ID NO: 1, SEQ ID NO: 3, SEQ ID NO:4, SEQ ID NO:5, SEQ ID NO:7, SEQ ID NO:8, SEQ ID NO:10, SEQ ID NO:12, SEQ ID NO:14, SEQ ID NO:16, SEQ ID NO:18, or SEQ ID NO:20. For example, the enzyme can be encoded by a nucleotide sequence that has an identity to the sequence set forth in SEQ ID NO: 1, SEQ ID NO: 3, SEQ ID NO:4, SEQ ID NO:5, SEQ ID NO:7, SEQ ID NO:8, SEQ ID NO:10, SEQ ID NO:12, SEQ ID NO:14, SEQ ID NO:16, SEQ ID NO:18, or SEQ ID NO:20 that is, is about, is less than, or is more than, 50%, 60%, 70%, 80%, 90%, 95%, 96%, 97%, 98%, 99%, 100%, or a value that is a range defined by any of these values (including the end points), for example, 90% to 100%, 95% to 99%, etc. In some embodiments, the enzyme has an amino acid sequence set forth in SEQ ID NO: 2, SEQ ID NO:6, SEQ ID NO:9, SEQ ID NO:11, SEQ ID NO:13, SEQ ID NO:15, SEQ ID NO:17, SEQ ID NO:19, or SEQ ID NO:21. In some embodiments, the enzyme has an amino acid sequence that is homologous to the sequence set forth in SEQ ID NO: 2, SEQ ID NO:6, SEQ ID NO:9, SEQ ID NO:11, SEQ ID NO:13, SEQ ID NO:15, SEQ ID NO:17, SEQ ID NO:19, or SEQ ID NO:21. For example, the enzyme has an amino acid sequence that has an identity to the sequence set forth in SEQ ID NO: 2, SEQ ID NO:6, SEQ ID NO:9, SEQ ID NO:11, SEQ ID NO:13, SEQ ID NO:15, SEQ ID NO:17, SEQ ID NO:19, or SEQ ID NO:21 that is, is about, is less than, or is more than, 50%, 60%, 70%, 80%, 90%, 95%, 96%, 97%, 98%, 99%, 100%, or a value that is a range defined by any of these values, for example, 90% to 100%, 95% to 99%, etc.

In some embodiments, it can be advantageous to specifically pair the acidifier (e.g., to an acidifier not adversely affecting the activity of the enzyme) with the enzyme. For example, the solid acidifier material can be specifically paired with the enzyme to adjust the environmental pH to enhance the activity of the enzyme. In some embodiments, the acidifier is in a solid form. The shape or form of the solid acidifier is not particularly limited. For example, the solid acidifier can be in the form of particle, powder, granular, crystalline, or any combination thereof. In some embodiments, the solid acidifier is in a crystalline form. In some embodiments, the solid acidifier is spherical or spheroid in nature. In some embodiments, the solid acidifier is in one or combinations of different geometric shapes, including but not limited to, cube, cuboid, cylinder, cone, prism, pyramid, and any other polygonal shapes. In some embodiments, the solid acidifier is in a fibrous form. In some embodiments, the solid acidifier material is in a powder form.

The enzymes disclosed herein (e.g., cellulases) may hydrolyze substrate polymers (e.g., guar polymers) at temperatures that are above 160° F. or 180° F. In some embodiments, the enzymes can hydrolyze the substrate polymers at temperatures in excess of 185° F. or 195° F. As would be appreciated by those of ordinary skill in the art, the enzymes may be used in combination with other enzymes and/or oxidative breakers to degrade substrate polymers (e.g., guar polymers) over broader temperature and pH ranges.

Additional Components

In addition to the enzyme and acidifying agent, the enzyme-containing core may include one or more additional components. Non-limiting examples of the additional component include binding agents, inert carriers, stabilizers, anti-tacking agents.

As used herein, the terms “binding agent” and “binders” are used interchangeably, and refer to any material capable of providing sufficient adhesion between various materials (e.g., the enzyme, the acidifying agent, the inert carrier, and/or the stabilizer). For example, the binding agent may sufficiently attach the enzyme to the acidifying agent so that the acidifying agent can serve as a carrier for the enzyme. In some embodiments, the binding agent attaches the enzyme on the outer surface of the acidifying agent.

The binding agent can comprise, or be, but not limited to, polyvinylpyrrolidone, polyvinyl alcohol, alginate, polyethylene glycol, wax (e.g., bee wax or synthetic wax), xanthan gum, polyvinyl acetate, carrageenans, starch, maltodextrin, hydroxypropyl cellulose, hydroxypropyl methylcellulose, methylcellulose, carboxymethyl cellulose, or any combination thereof.

The amount of binding agent in the enzyme-containing core can vary. For example, the amount of the binding agent in the enzyme-containing core can be, or be about 0.1%, 0.5%, 1%, 2%, 3%, 4%, 5%, 6%, 7%, 8%, 9%, 10%, 15%, 20%, 30%, 40%, or 50%, or any range between two of these values by weight, based on the total weight of the enzyme-containing core. In some embodiments, the binding agent is present in the enzyme-containing core at an amount of about 0.1% to about 10% based on the total weight of the enzyme-containing core.

In some embodiments, the enzyme-containing core comprises one or more inert carriers. Examples of inert carriers include, but are not limited to, fibrous and microcrystalline cellulose, sodium sulfate, sodium chloride, monocalcium phosphate, dicalcium phosphate, tricalcium phosphate, monosodium phosphate, disodium phosphate, trisodium phosphate, monopotassium phosphate, dipotassium phosphate, tripotassium phosphate, calcium carbonate, diatomaceous earth, zeolite, starch, and any combination thereof.

In some embodiments, the enzyme-containing core comprises one or more stabilizers. Examples of stabilizers include, but are not limited to, mannitol, trehalose, sorbitol, xylitol, sucrose, microcrystalline cellulose, starch, sodium chloride, sodium sulfate, ammonium sulfate, and any combination thereof. The stabilizer can be multifunctional. For example, in some embodiments, the stabilizer can have properties to function as an acidifying agent and/or a binder.

### Shells

As disclosed herein, the particle for well treatment can comprise a shell configured to at least partially encapsulate the enzyme-containing core. The shell can, in some embodiments, allow immediate, controlled, and/or sustained release of the acidifying agent and/or the enzyme encapsulated in the shell. In some embodiments, the shell allows controlled release of the acidifying agent and/or the enzyme encapsulated in the shell. For example, the shell can include one or more agents that are breakable and/or soluble in the environment (e.g., a subterranean treatment fluid) in which the particles are present.

The extent to which the shell encapsulates the enzyme-containing core can vary. For example, the shell can partially cover the surface area of the enzyme-containing core, or substantially cover the entire surface area of the enzyme-containing core. In some embodiments, the shell covers about 5%, 10%, 15%, 20%, 30%, 40%, 50%, 60%, 70%, 80%, 90%, 95%, 99%, 100%, or a range between any two of these values (including the end points) of the surface area of the enzyme-containing core. In some embodiments, the shell covers at least, or at least about 5%, 10%, 15%, 20%, 30%, 40%, 50%, 60%, 70%, 80%, 90%, 95%, 99%, 100% of the surface area of the enzyme-containing core. In some embodiments, the shell covers the entire surface area of the enzyme-containing core. In some embodiments, the shell substantially encapsulates the enzyme-containing core. In some embodiments, the shell encapsulates the entire enzyme-containing core.

The breaking and/or dissolution of the shell, or a portion thereof, in some embodiments, can lead to the exposure of the enzyme-containing core to the environment (e.g., a subterranean treatment fluid) in which the particles of well treatment are present. In some embodiments, it is possible to control the release of the enzyme-containing core based on the chemical composition or physical properties of the shell. In some embodiments, the thickness of the shell is correlated with the permeability of the shell. In some embodiments, the thickness of the shell is correlated with the time needed for the shell to break or dissolve to the extent that allows the enzyme-containing core to be exposed to the environment (e.g., a subterranean treatment fluid) in which the particles of well treatment are present. In some embodiments, it takes at least about 1 minute, about 10 minutes, about 30 minutes, about 1 hour, about 5 hours, about 8 hours, about 10 hours, about 11 hours, about 12 hours, about 13 hours, about 14 hours, about 15 hours, about 16 hours, about 17 hours, about 18 hours, about 19 hours, about 20 hours, about 25 hours, about 30 hours, about 40 hours, or about 50 hours, or a range between any two of these values, or longer, for the shell to break or dissolve to the extent that allows the enzyme-containing core to be exposed to the environment (e.g., a subterranean treatment fluid) in which the particles of well treatment are present, and triggers the enzyme to become active. In some embodiments, it takes about 30 minutes to about 2 hours for the shell to break or dissolve to the extent that allows the enzyme-containing core to be exposed to the environment (e.g., a subterranean treatment fluid) in which the particles of well treatment are present, and triggers the enzyme to become active.

Co-encapsulation of acidifying agent (e.g., one or more solid acidifiers) and one or more enzymes can be advantageous as compared to the previously known compositions and methods for breaking viscosified gelled fluids. For example, the co-encapsulation of enzyme breaker and acidifier can, in some embodiments, allow ascertain the ratio of enzyme to acidifier, as opposed to separately added acidifying agents which may vary in concentration and distribution may cause incomplete break in some areas. Additionally, unlike separately added acidifying and breaker agents, the co-encapsulated enzyme and acidifier can, in some embodiments, be distributed in a desired pattern (e.g., equally) within the subterranean formation. In some embodiments, the enzyme and the acidifier can contact the environment at similar rates and at similar times as both are contained within the same encapsulating material thereby creating a localized environment that may result in prolonged enzyme activity.

Without being bound by any particular theory, it is believed that the shells disclosed herein can function, in some embodiments, as protective coatings for the enzyme-containing core. It is advantageous, in some embodiments, that the shells are thermally stable and do not degrade initially upon contact with the environment (e.g., a subterranean treatment fluid) in which the particles of well treatment are present. Various factors can be considered in determining the delay of contact between the enzyme breaker and the substrate in the environment (e.g., viscosified fracturing fluids). Non-limiting examples of these factors include the type of the enzyme breaker, chemical and/or physical properties of the enzyme breaker, environmental conditions, chemical and/or physical properties of the shell, and concentration used.

The eventual loss of integrity of the encapsulant material and contact of the enzyme breaker with the viscosified subterranean treatment fluid may occur through one or more mechanisms including, but are not limited to, direct dissolution (e.g., direct dissolution of the enzyme into the surrounding fluid through incomplete encapsulating shell coverage), diffusion (e.g., diffusion of the enzyme molecules through the pores of the shell into the surrounding fluid), degradation, biodegradation (e.g., biodegradation of the encapsulation shell that allows the exposure of the enzyme to the surrounding fluid), swelling, melting, disintegration, fragmentation (e.g., fragmentation of the encapsulating shell, disintegration and/or fragmentation of the particles), and the like. In some embodiments, the contact of the enzyme breaker with the viscosified fracturing fluid may also occur by diffusion of the enzymes through small pores without removal of the shell or a portion thereof. In some embodiment, solvents (e.g., water molecules) from the surrounding fluid can diffuse to inside the shell or into the enzyme-containing core through pores, incomplete shell coverage, degradation and/or biodegradation of the shell, melting of the shell. In some embodiments, the solvent molecules can dissolve the enzyme from the core, and the dissolved enzyme molecules can diffuse to the surrounding fluid through pores or other openings on the shell. In some embodiments, when water diffuses into the shell, the hydrated shell and/or enzyme-containing cores can swell and result in disintegration and/or fragmentation of the shell. In some embodiments, the delay can correspond to a certain event or combination of events, for example exposure to high temperature and pressure at which point a reduction in viscosity may be desirable. As would be appreciated by one of ordinary skill in the art, the release of the acidifying agents into the surrounding fluid can be through similar or different mechanism(s) as the release of the enzyme.

The encapsulated enzyme break compositions disclosed herein can be used to break, for example subterranean treatment fluids, at relatively high temperature ranges (e.g., between 150° F. to 200° F.). For example, the formulated enzyme breakers disclosed herein may break the subterranean treatment fluids at a temperature that is, is about, is less than, or is more than, ambient, 80° F., 100° F., 120° F., 140° F., 160° F., 170° F., 180° F., 190° F., 195° F., 200° F., 205° F., 210° F., 215° F., 220° F., 230° F., 240° F., or a value that is a range defined by any of these values (including the end points), for example, 140° F. to 180° F., 120° F. to 160° F., etc. In some embodiments, the formulated enzyme breakers can break the subterranean treatment fluids at a temperature that is greater than about 140° F. In some embodiments, the formulated enzyme breakers can break the subterranean treatment fluids at a temperature that is greater than about 180° F. In some embodiments, the formulated enzyme breakers can break the subterranean treatment fluids at a temperature that is greater than about 195° F. It is believed that these temperatures are significantly higher than the workable temperature ranges for conventional encapsulated enzyme breakers, which represents a distinct advantage.

The shell can comprises one or more encapsulant materials. It can be advantageous, in some embodiments, to use encapsulant materials that do not adversely interact or chemically react with the enzyme to destroy its utility. For example, the encapsulant materials can comprise, or be, polymers, homopolymers, copolymers, or any combination thereof. As used herein, the term “copolymer” refers to a polymer derived from more than one species of monomer. The type of the copolymer can vary. Non-limiting examples of copolymer include bipolymers (i.e., polymers that are obtained by copolymerization of two monomer species), terpolymers (i.e., polymers that are obtained from three species of monomers), and quaterpolymers (i.e., polymers that are obtained from four species of monomers). The copolymer can be, for example, alternating copolymers, periodic copolymers, statistical copolymers, block copolymers, or any combination thereof. In some embodiments, the copolymers can be linear polymers, branched polymers, polymers with both linear and branched portions, or any combination thereof. In some embodiments, the shell comprises one or more homopolymers, one or more copolymers, or any combination thereof.

In some embodiments, the encapsulant material comprises or is ethylcellulose, acrylic resin, nitrocellulose, plastics, methacrylate, acrylic acetate, polyvinylidene chloride (PVDC), polyurethane, wax, polyethylene, polyethylene glycol, polyvinylalcohol, polyester, polylactic acid, polyglycolic acid, copolymers of polylactic and polyglycolic acids, polyvinyl acetate, vinyl acetate acrylic copolymer, or any combination thereof. In some embodiments, the shell comprises one or more polymers (homopolymers or copolymers) comprising one or more of the monomers selected from the group consisting of methacrylic acid, methacrylic ester, methacrylic amide, methacrylic nitril, acrylic acid, acrylic ester, acrylic amide, acrylic nitril, styrene/n-butyl acrylate copolymer, and vinyl monomers. In some embodiments, the shell comprises one or more polymers (homopolymers or copolymers) made from methacrylic acid, methacrylic ester, methacrylic amide, methacrylic nitril, acrylic acid, acrylic ester, acrylic amide, acrylic nitril, vinyl monomers, or any combination thereof. Vinyl monomers can include, but are not limited to, styrene monomers, alpha methyl styrene monomers, and any combination thereof. In some embodiments, the shell comprises one or more polymers (homopolymers or copolymers) derived from methacrylic acid, methacrylate, acrylic acid, acrylate, vinyl monomers (for example, styrene and alpha methyl styrene), or any combination thereof.

Additional non-limiting examples of the encapsulant material include vinyl-acrylic emulsion, polyvinyl acetate dispersion, acrylic emulsion polymer, aqueous dispersion of styrene-acrylate copolymer, aqueous dispersion of styrene/n-butyl acrylate copolymer, aqueous dispersion of anionic polyurethane, and any combination thereof. As used herein, the terms “dispersion” and “emulsion” are used interchangeably.

The formulated enzyme breakers disclosed herein can also comprise one or more anti-tacking agents. Without being limited to any particular theory, it is believed that some encapsulant materials are sticky in nature, which may lead to the agglomeration of the enzyme-containing core particles (e.g., several core particles are glued together) during the coating process to form the shell. Thus, it may be advantageous, in some embodiments, to apply anti-tacking agent(s) during the process of coating the enzyme-containing cores to minimize the agglomeration of enzyme-containing cores, so that each enzyme-containing core can be coated individually. Various techniques can be used to apply the anti-tacking materials. For example, the anti-tacking materials can be added as powder when the shell material is sprayed onto the enzyme-containing cores. As another example, the anti-tacking materials can be added into a solution or dispersion of the encapsulant material to form a mixture, and sprayed the mixture onto the enzyme-containing core to form a shell. In some embodiments, the anti-tacking agent(s) are imbedded within and/or present on the surface of the encapsulating shell layer. Examples of anti-tacking agents include, but not limited to talc, silica dioxide, calcium stearate, zinc stearate, magnesium stearate, diatomaceous earth, kaolin, bentonite, and any combinations thereof.

The size of the particles for well treatment can vary, for example, from about 7 mesh to about 60 mesh on the U.S. Sieve Series. For example, the particles for well treatment can be about 2.8 mm to about 0.25 mm. For example, the size of the particle can be about, or is, 7 mesh (2.8 mm), 8 mesh (2.4 mm), 10 mesh (2 mm), 12 mesh (1.7 mm), 14 mesh (1.4 mm), 16 mesh (1.2 mm), 18 mesh (1 mm), 20 mesh (0.84 mm), 30 mesh (0.59 mm), 35 mesh (0.5 mm), 40 mesh (0.42 mm), 45 mesh (0.35 mm), 50 mesh (0.3 mm), 60 mesh (0.25 mm) on the U.S. Sieve Series, or a value between any two of these values (including the end points). In some embodiments, the average size of a plurality of the particles is, or is about, 7 mesh, 8 mesh, 10 mesh, 12 mesh, 14 mesh, 16 mesh, 18 mesh, 20 mesh, 25 mesh, 30 mesh, 35 mesh, 40 mesh, 45 mesh, 50 mesh, 60 mesh on the U.S. Sieve Series, or a value between any two of these values (including the end points). In some embodiments, the size of the particle is, or is about, 7-60 mesh, 18-60 mesh, 20-50 mesh, 30-40 mesh, 8-40 mesh, 8-30 mesh, 8-20 mesh, 8-18 mesh, 10-30 mesh, 10-25 mesh, 10-20 mesh, 10-18 mesh, 12-30 mesh, 12-25 mesh, 12-20 mesh, or 12-18 mesh on the U.S. Sieve Series. In some embodiments, the average size of a plurality of the particles is 7-60 mesh, 18-60 mesh, 20-50 mesh, 30-40 mesh, 8-40 mesh, 8-30 mesh, 8-20 mesh, 8-18 mesh, 10-30 mesh, 10-25 mesh, 10-20 mesh, 10-18 mesh, 12-30 mesh, 12-25 mesh, 12-20 mesh, or 12-18 mesh on the U.S. Sieve Series. In some embodiments, the size of the particle is about 7 mesh to about 60 mesh on the U.S. Sieve Series. In some embodiments, the size of the particle is about 10 mesh to about 20 mesh on the U.S. Sieve Series.

The particle for well treatment disclosed herein can also comprise one or more additional layers of coatings (e.g., successive layers of coatings) outside of or underneath the shell. It can be advantageous, in some embodiments, to have one or more layers of coatings to provide further protection (e.g., chemical or physical protection) to the enzyme to avoid reduction or loss of enzyme activity, to prevent undesirable leak of enzyme from the particles. The one or more layers of coating can also, for example, functions as polish coating(s) to improve shell life, easy of handling, prevent compression, and/or appearance of the particle. Different layer of coating may serve different functions, including but not limited to, delaying enzyme release (i.e., release layer), protecting the enzyme from the environment and incompatible encapsulant materials (protective layer), and improving production process and handling properties (polish layer). For example, the additional layer of coating may act to delay activity of the acidifier and/or enzyme for differing periods of time. In some embodiments, at least one of the additional layers of coating is a polymeric protective layer. In some embodiments, at least one of the additional layers of coating is a polymeric polish layer. In some embodiments, the particle comprises at least one of a release layer, a protective layer, and a polish layer. In some embodiments, the polish layer is the outer most layer of the particle. In some embodiments, the polish layer is outside of the protective layer and/or the release layer. In some embodiments, the protective layer is underneath the release layer. For example, the protective layer can be inside of the release layer in the particle.

A protective layer can comprise any one or a combination of the binding agents disclosed herein. In some embodiments, the protective layer comprises polyvinylpyrrolidone, polyvinyl alcohol, alginate, polyethylene glycol, wax (e.g., bee wax or synthetic wax), xanthan gum, polyvinyl acetate, starch, maltodextrin, carrageenans, hydroxypropyl cellulose, hydroxypropyl methylcellulose, methylcellulose, carboxymethyl cellulose, or any combination thereof. A polish layer can also comprise any one or a combination of the binding agents or encapsulant polymers disclosed herein. In some embodiments, the polish layer comprises polyvinylpyrrolidone, polyvinyl alcohol, alginate, polyethylene glycol, wax (e.g., bee wax or synthetic wax), xanthan gum, polyvinyl acetate, starch, maltodextrin, carrageenans, hydroxypropyl cellulose, hydroxypropyl methylcellulose, methylcellulose, carboxymethyl cellulose, ethylcellulose, nitrocellulose, acrylic resin, plastics, methacrylate, acrylic acetate, polyvinylidene chloride (PVDC), polyurethane, polyethylene, polyester, polylactic acid, polyglycolic acid, copolymers of polylactic and polyglycolic acids, vinyl acetate acrylic copolymer, styrene-acrylate copolymer, styrene/n-butyl acrylate copolymer, polymers (homopolymers or copolymers) derived from methacrylic acid, methacrylate, acrylic acid, acrylate, vinyl monomers (for example, styrene and alpha methyl styrene), or any combination thereof.

Various additional components can also be present in the particles for well treatments, including but are not limited to oxidizing agents. One or more oxidizing agents can be present in any portion of the particles, for example, the enzyme-containing core, the shell surrounding the enzyme-containing core, any of the one or more additional layers of coatings outside of or underneath the shell, or any combination thereof. It may be advantageous, in some embodiments, to avoid contacts of the enzyme with the oxidizing agent which may degrade and destabilize the enzyme during long term storage. In some embodiments, the oxidizing agent is separated from the enzyme-containing core by at least one layer of coating or shell that does not contain the oxidizing agent. For example, the particle can comprise an enzyme-containing core surrounded by a non-oxidizing agent-containing shell, and a layer of oxidizing agent-containing coating outside of the shell. In some embodiments, the oxidizing agent is sequestered from the enzyme (e.g., by a layer of coating) within the enzyme-containing core. For example, the particle can comprise one or more oxidizing agents that are coated by one or more layers of polymer coating, and dispersed within and/or present at the outer surface of an enzyme containing core.

Without being limited by any particular theory, it is believed that, in some embodiments, it is advantageous to have different components serve different purposes in the particles for well treatment, for example, the enzyme functions as the breaker and the acidifying agent functions as a pH-adjusting agent.

### Methods for Making Particles for Well Treatment

Also provided herein are methods for making particles for well treatment.

In some embodiments, the method comprises contacting an enzyme with a solid acidifying agent to form an enzyme-containing core; and encapsulating the enzyme-containing core with one or more shells to form a particle for well treatment, wherein each of the one or more shells is configured to at least partially encapsulate the enzyme-containing core.

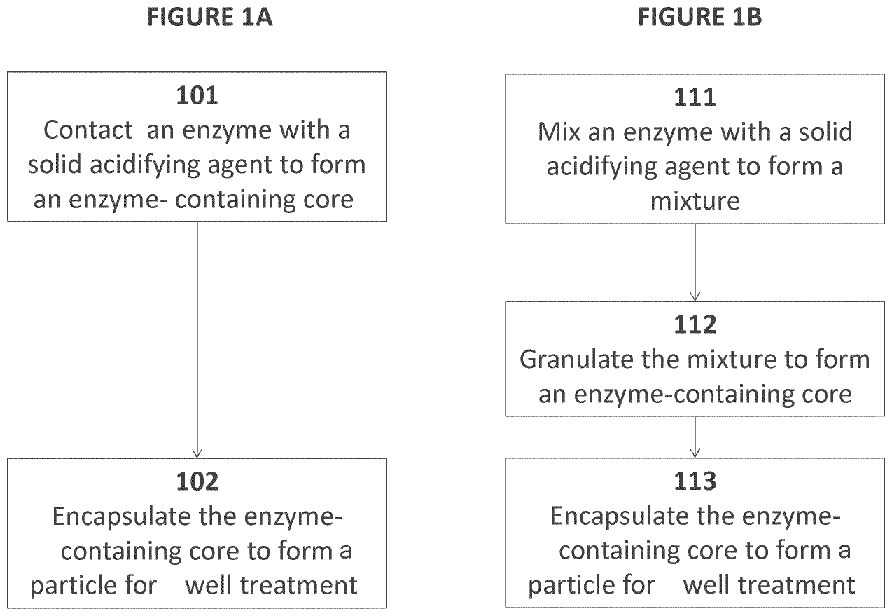

FIG. 1A depicts an illustrative embodiment of the method within the scope of the present disclosure. Beginning at block 101 (Contact an enzyme with a solid acidifying agent to form an enzyme-containing core), an enzyme and a solid acidifying agent are provided and contacted with each other to form an enzyme-containing core. In some embodiments, the enzyme is attached to the solid acidifying agent, for example by a non-perforated pan coating process. a pan coating process, a fluidized bed coating process, a spray drying process, a continuous coating process, or any combination thereof. Non-limiting examples of the fluidized bed coating process include a bottom spray process, a Wurster process, a top spray process, a tangential spray process, a spouted bed process, a modified fluidized bed coating process. Other methods to attach the enzyme to the solid acidifying agent include using spray nozzle(s) mounted on top of or in a mixer, such as a ribbon blender. In some embodiments, contacting the enzyme with the solid acidifying agent comprises spraying a solution comprising the enzyme onto the solid acidifying agent. The enzyme can be any of those discussed above with respect to the particles for well treatment. For example, the enzyme can be a cellulase, a hemicellulase, a pectinase, a xanthanase, a mannanase, a galactosidase, a glucanase, an amylase, an amyloglucosidase, an invertase, a maltase, an endoglucanase, a cellobiohydrolase, a glucosidase, a xylanase, a xylosidase, an arabinofuranosidase, an oligomerase, or any combination thereof. In some embodiments, the enzyme is a thermostable or thermotolerant enzyme. The acidifying agent can be any of those discussed above with respect to the particles for well treatment. For example, the acidifying agent can comprise an inorganic acid, an organic acid, a salt or ester thereof, or any combination thereof. In addition to the enzyme and the acidifying agent, the enzyme-containing core can also include one or more other components, such as binding agents, inert carriers, stabilizers, or any other of the components described above with respect to the enzyme-containing core. In some embodiments, the acidifying agent comprises an organic buffer, including but not limited to a Tris-HCl buffer, a morpholino-ethanesulphonic acid (MES) buffer, a pyridine, cacodylate buffer, a bis(2-hydroxyethyl)amino-tris(hydroxymethyl)methane (BIS-TRIS( ) buffer, a piperazine-N,N′-bis(2-ethanesulfonic acid (PIPES) buffer, a 3-(N-morpholino)propanesulfonic acid (MOPS) buffer, a 3-(N-Morpholino)-2-hydroxypropanesulfonic acid (MOPSO) buffer, an ethylene-diamine-tetraacetic acid (EDTA) buffers, a glycine buffer, and any combination thereof.

In some embodiments, the acidifying agent, the binding agent, the inert carrier, the stabilizer, and/or any of the other component(s) included in the enzyme-containing core can be grinded (separately or together) before, during or after being combined to form the enzyme-containing core. As would be appreciated by those of ordinary skill in the art, various grinding mediums can be used during the grinding process as long as the grinding medium does not react with the acidifying agent, the binding agent, the inert carrier, the stabilizer, and/or any of the other component(s) included in the enzyme-containing core. In some embodiments, the acidifying agent, the binding agent, the inert carrier, the stabilizer, and/or any of the other component(s) included in the enzyme-containing core can be sieved (separately or together) before or during being combined to form the enzyme-containing core. In some embodiments, the enzyme-containing cores can be further sorted according to their sizes. For example, the enzyme-containing cores can be sieved and only enzyme-containing cores of certain sizes are retained. Block 101 may be followed by block 102.

At block 102 (Encapsulate the enzyme-containing core to form a particle for well treatment), the enzyme-containing core is encapsulated with one or more shells to form a particle for well treatment and each of the shells is configured to at least partially encapsulate the enzyme-containing core. The enzyme-containing core can be encapsulated by one, two, three, four, five, six, seven, eight, nine, ten, or more layers of shells. In some embodiments, the enzyme-containing core is encapsulated by successive shells. In some embodiments, at least two of the shells overlap with each other.

In some embodiments, the method comprises mixing an enzyme with a solid acidifying agent to form a mixture; granulating the mixture to form an enzyme-containing core; and encapsulating the enzyme-containing core with one or more shells to form a particle for well treatment, wherein each of the one or more shells is configured to at least partially encapsulate the enzyme-containing core.

FIG. 1B depicts an illustrative embodiment of the method within the scope of the present disclosure. Beginning at block 111 (Mix an enzyme with a solid acidifying agent to form a mixture), an enzyme and a solid acidifying agent are provided and mixed with each other to form a mixture. The enzyme can be any of those discussed above with respect to the particles for well treatment. For example, the enzyme can be a cellulase, a hemicellulase, a pectinase, a xanthanase, a mannanase, a galactosidase, a glucanase, an amylase, an amyloglucosidase, an invertase, a maltase, an endoglucanase, a cellobiohydrolase, a glucosidase, a xylanase, a xylosidase, an arabinofuranosidase, an oligomerase, or any combination thereof. In some embodiments, the enzyme is a thermostable or thermotolerant enzyme. The acidifying agent can be any of those discussed above with respect to the particles for well treatment. For example, the acidifying agent can comprise an inorganic acid, an organic acid, a salt or ester thereof, or any combination thereof. In addition to the enzyme and the acidifying agent, the mixture can, in some embodiments, include one or more other components, such as binding agents, inert carriers, stabilizers, or any other of the components described above with respect to the enzyme-containing core.

In some embodiments, the acidifying agent, the binding agent, the inert carrier, the stabilizer, and/or any of the other component(s) included in the mixture can be grinded (separately or together) before, during or after being combined to form the mixture. As would be appreciated by those of ordinary skill in the art, various grinding mediums can be used during the grinding process as long as the grinding medium does not react with the acidifying agent, the binding agent, the inert carrier, the stabilizer, and/or any of the other component(s) included in the mixture. In some embodiments, the acidifying agent, the binding agent, the inert carrier, the stabilizer, and/or any of the other component(s) included in the mixture can be sieved (separately or together) before or during being combined to form the mixture. Block 111 may be followed by block 112.

At block 112 (Granulate the mixture to form an enzyme-containing core), the mixture is granulated to form enzyme-containing cores. The mixture can be granulated using any granulation techniques known in the art, for example, a wet granulation process. In some embodiments, the wet granulation process comprises extrusion, centrifugal extrusion, spheronization, batch high shear granulation, continuous high shear mixing, disc granulation, drum granulation, spray drying, fluid bed agglomeration, fluid bed granulation and/or layering (e.g., bottom spray, tangential spray, and spouted bed), prilling, or any combination thereof. In some embodiments, the enzyme-containing cores can be further sorted according to their sizes. For example, the enzyme-containing cores can be sieved and only enzyme-containing cores of certain sizes are retained. Block 112 may be followed by block 113.

At block 113 (Encapsulate the enzyme-containing core to form a particle for well treatment), the enzyme-containing core is encapsulated with one or more shells to form a particle for well treatment and each of the shells is configured to at least partially encapsulate the enzyme-containing core. In some embodiments, the enzyme-containing core is dried before being encapsulated by the one or more shell. The enzyme-containing core can be encapsulated by one, two, three, four, five, six, seven, eight, nine, ten, or more layers of shells. In some embodiments, the enzyme-containing core is encapsulated by successive shells. In some embodiments, at least two of the shells overlap with each other.

As described herein, for example at blocks 102 and 113, the enzyme-containing core can be coated with the one or more shells using any suitable methods known in the art, for example encapsulation or microencapsulation techniques. In some embodiments, the enzyme-containing core is encapsulated using a non-perforated pan coating process, a pan coating process, a fluidized bed coating process, a spray drying process, a continuous coating process, or any combination thereof. Non-limiting examples of the fluidized bed process include a bottom spray process, a Wurster process, a top spray process, a tangential spray process, a spouted bed process, a modified fluid bed coating process, or any combination thereof. In some embodiments, the encapsulation is achieved using spray nozzle(s) mounted on top of or in a mixer, such as a ribbon blender. A spray drying process may also be used as a suitable encapsulation technique. In some embodiments, the enzyme-containing core is encapsulated using a pan coating technique.

Each of the shells is configured to at least partially encapsulate the enzyme-containing core. For example, a shell may cover substantially all of the surface area of the enzyme-containing core, or only a portion. In some embodiments, the shell covers all of the surface area of the enzyme-containing core, and thus fully encapsulates the reactive material. All, or a portion, of the total enzyme-containing core may be coated with the shell. For example, about 10%, about 20%, about 30%, about 40%, about 50%, about 60%, about 70%, about 80%, about 90%, about 95%, or about 100%, or a range between any two of these values, of the enzyme-containing core may be coated with each of the shells. The extent by which each of the shells encapsulates the enzyme-containing core can vary. In some embodiments, all of the shells cover substantially similar portion of the surface area of the enzyme-containing core. In some embodiments, at least two of the shells cover different portion of the surface area of the enzyme-containing core. In some embodiments, the shell comprises ethylcellulose, acrylic resin, plastics, methacrylate, acrylate, acrylic acetate, polyurethane, polyvinylidene chloride (PVDC), nitrocellulose, wax, polyethylene, polyethylene glycol, polyvinylalcohol, polyester, polylactic acid, polyglycolic acid, copolymers of polylactic and polyglycolic acids, polyvinyl acetate, vinyl acetate acrylic copolymer, alginates, agar, styrene acrylic copolymer, styrene/n-butyl acrylic copolymer or any combination thereof.

Each of the shells can be the same or different in composition or thickness. In some embodiments, all of the shells have the same composition. In some embodiments, at least two of the shells have different composition. In some embodiments, all of the shells have different composition from each other. In some embodiments, the thicknesses of all of the shells are the same. In some embodiments, at least two of the shells have different thickness. In some embodiments, all of the shells have different thickness.

The extent by which each particle made by the methods disclosed herein has the shell covered on its surface can also vary. In some embodiments, about 10%, about 20%, about 30%, about 40%, about 50%, about 60%, about 70%, about 80%, about 90%, about 95%, or about 100%, or a range between any two of these values, of the particles have substantially all their surface areas covered by the shell. In some embodiments, about 10%, about 20%, about 30%, about 40%, about 50%, about 60%, about 70%, about 80%, about 90%, about 95%, or about 100%, or a range between any two of these values, of the particles have at least about 50%, 60%, 70%, 80%, 90%, 95%, 99%, or more of all their surface areas covered by the shell. It will be appreciated by those of ordinary skill in the art that the technique(s) in which the encapsulation (e.g., at blocks 102 and 113) is achieved is not limited in any way. In some embodiments, the encapsulation step includes spraying and/or drying (e.g., thermal current drying). In some embodiments, the particles are cured at an elevated temperature to promote formation of at least one of the shells (e.g., film formation of the shell(s)). In some embodiments, the elevated temperature for curing is, or is about, 25° C., 30° C., 35° C., 40° C., 45° C., 50° C., 55° C., 60° C., 65° C., 70° C., 75° C., 80° C., or a range between any two of these values (including the end points). In some embodiments, the elevated temperature for curing is between about 25° C. to about 80° C. In some embodiments, the elevated temperature for curing is between about 40° C. to about 60° C.

The weight gain for the enzyme-containing cores as a result of the encapsulation process can vary. The weight gain can be measured, for example, by the theoretical percentage increase of dry, encapsulated product weight from the original core subsequent to the coat application. Without being bound by any particular theory, it is believed that the weight gain is indicative of coating thickness. For example, the weight gain for the enzyme-containing cores after drying off water can be about 10% to about 250%. In some embodiments, the weight gain is, or is about, 10%, 20%, 30%, 40%, 50%, 60%, 70%, 80%, 90%, 100%, 110%, 120%, 130%, 140%, 150%, 160%, 170%, 180%, 190%, 200%, or a range between any two of these values. In some embodiments, the weight gain for the enzyme-containing cores after drying off water is about 100%. In some embodiments, the weight gain for the enzyme-containing cores after drying off water is about 20% to about 250%, about 40% to about 200%, about 50% to about 150%, or about 80 to about 120%. In some embodiments, the weight gain after drying off water is 100%.

The size of the particles made by the methods disclosed herein is not limited in any way. For example, the average size of the particles can be, or be about, 7 mesh, about 8 mesh, about 10 mesh, about 12 mesh, about 14 mesh, about 16 mesh, about 18 mesh, about 20 mesh, about 25 mesh, about 30 mesh, about 35 mesh, about 40 mesh, about 45 mesh, about 50 mesh, about 60 mesh on the U.S. Sieve Series, or a range between any two of these values (including the end points). In some embodiments, the average size of the particles is about 7 mesh to about 60 mesh, about 18 mesh to about 60 mesh, about 20 mesh to about 50 mesh, about 30 mesh to about 40 mesh, about 8 mesh to about 40 mesh, about 8 mesh to about 30 mesh, about 8 mesh to about 20 mesh, about 8 mesh to about 18 mesh, about 10 mesh to about 30 mesh, about 10 mesh to about 25 mesh, about 10 mesh to 20 mesh, about 10 mesh to about 18 mesh, about 12 mesh to about 30 mesh, about 12 mesh to about 20 mesh, about 12 mesh to about 25 mesh, or about 12 mesh to about 18 mesh on the U.S. Sieve Series. In some embodiments, the particles can be further sorted according to their sizes. For example, the particles can be sieved and only particles of certain sizes are retained. In some embodiments, the particles with the size of 7 mesh to 60 mesh on the U.S. Sieve Series are retained. In some embodiments, the particles with the size of 10 mesh to 20 mesh on the U.S. Sieve Series are retained.

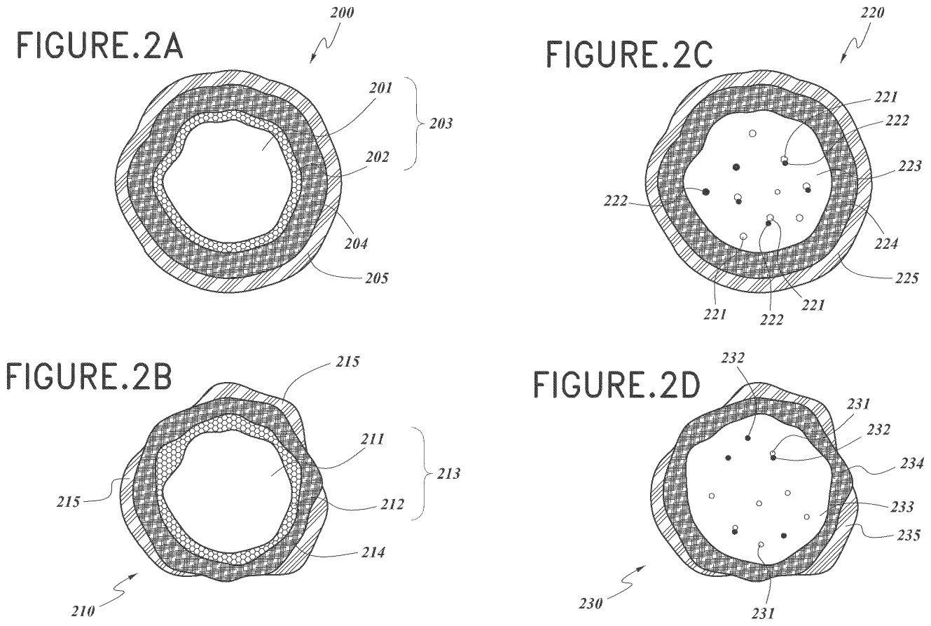

FIG. 2A shows a cross-section of a non-limiting embodiment of a particle 200 for well treatment. In particle 200, an enzyme-containing core 203 is encapsulated with a shell 204 and an optional shell 205. Each of shells 204 and 205 covers substantially the entire surface area of the enzyme-containing core 203. Enzyme-containing core 203 contains a solid acidifying agent 201 and an enzyme 202, and enzyme 202 is present substantially on the surface of the enzyme-containing core 203. FIG. 2B shows a cross-section of another non-limiting embodiment of a particle 210 for well treatment. In particle 210, an enzyme-containing core 213 is encapsulated with a shell 214 and an optional shell 215. Shell 214 covers substantially the entire surface area of the enzyme-containing core 213, and Shell 215 covers only a portion of the surface area of the enzyme-containing core 213. The enzyme-containing core 213 contains a solid acidifying agent 211 and an enzyme 212, and enzyme 212 is present substantially on the surface of the enzyme-containing core 213.

FIG. 2C shows a cross-section of a non-limiting embodiment of a particle 220 for well treatment. In particle 220, an enzyme-containing core 223 is encapsulated with a shell 224 and an optional shell 225. Each of shells 224 and 225 covers substantially the entire surface area of the enzyme-containing core 223. The enzyme-containing core 223 contains a solid acidifying agent 221 and an enzyme 222 that are dispersed within the enzyme-containing core 223. FIG. 2D shows a cross-section of another non-limiting embodiment of a particle 230 for well treatment. In particle 230, an enzyme-containing core 233 is encapsulated with a shell 234 and an optional shell 235. Shell 234 covers substantially the entire surface area of the enzyme-containing core 233, and Shell 235 covers only a portion of the surface area of the enzyme-containing core 233. The enzyme-containing core 233 contains a solid acidifying agent 231 and an enzyme 232 that are dispersed within the enzyme-containing core 233. In particles 220 and 230, one or more of the solid acidifying agents 221 and 231, and enzymes 222 and 232 can be randomly dispersed or dispersed in a predetermined pattern within the enzyme-containing cores 223 and 233.

In a non-limiting example of encapsulation process, a solution comprising an enzyme and a binder is sprayed to acidifying core particles to form enzyme-containing cores. The enzyme-containing cores are dried and sprayed with a solution or dispersion comprising an encapsulant material to form a shell to partially or entirely encapsulate the enzyme-containing cores. The resulting encapsulated particles can then be dried to form encapsulated enzyme breakers. One or more additional shells can also be added to the encapsulated breakers.

Also disclosed are compositions that comprise any of the particles for well treatment disclosed herein. The form of the compositions is not particularly limited. For example, the composition can be in a solution or an aqueous dispersion. In some embodiments, the composition comprising particle(s) for well treatment is in a solution. In some embodiments, the composition comprising particle(s) for well treatment is in an aqueous dispersion.

As disclosed herein, the formulated enzyme breakers disclosed herein can comprise any of the particles for well treatment disclosed herein, or any combination of the particles. The formulated enzyme breakers can, for example, break substrates in target compositions that are involved in hydrocarbon recovery processes. Examples of the target compositions include, but are not limited to, fracturing fluids, drilling fluids, gravel packing fluids, completion fluids, workover fluids, filter cakes, and any combination thereof.

The formulated enzyme breaker can be configured, in some embodiments, to break the substrates in target compositions in a controlled manner. For example, the formulated enzyme breaker may exhibit a delayed enzyme release pattern. In some embodiments, it can take a time period that is, or is about, 1 minute, 5 minutes, 10 minutes, 15 minutes, 20 minutes, 30 minutes, 40 minutes, 50 minutes, 1 hour, 1.5 hour, 2 hours, 4 hours, 8 hours, 12 hours, 16 hours, 20 hours, 24 hours, 36 hours, 48 hours, or a range between any two of these values (including the end points) for the formulated enzyme breaker to release about 5%, about 10%, about 20%, about 30%, about 40%, about 50%, about 60%, about 70%, about 80%, about 90%, about 95%, about 99%, about 100%, or a range between any two of these values of the enzyme present in the formulated enzyme breaker to the target composition from the time that the formulated enzyme breaker becomes in contact with the target composition. In some embodiments, it takes at least about 30 minutes for the formulated enzyme breaker to release about 50%, about 60%, about 70%, about 80%, about 90%, about 95%, about 99%, about 100%, or a range between any two of these values of the enzyme present in the formulated enzyme breaker to the target composition from the time that the formulated enzyme breaker becomes in contact with the target composition. In some embodiments, it takes about 1 hour for the formulated enzyme breaker to release about 50%, about 60%, about 70%, about 80%, about 90%, about 95%, about 99%, about 100%, or a range between any two of these values of the enzyme present in the formulated enzyme breaker to the target composition from the time that the formulated enzyme breaker becomes in contact with the target composition. In some embodiments, it takes about 15 minutes to about 24 hours, about 20 minutes to about 8 hours, about 30 minutes to about 4 hours, about 40 minutes to about 2 hours for the formulated enzyme breaker to release about 50%, about 60%, about 70%, about 80%, about 90%, about 95%, about 99%, about 100%, or a range between any two of these values of the enzyme present in the formulated enzyme breaker to the target composition from the time that the formulated enzyme breaker becomes in contact with the target composition. Persons of ordinary skill in the art will be able to determine the desired delay time according to factors including but not limited to, desired use, well conditions, composition of the target composition (e.g., fracturing fluids, drilling fluids, completion fluids, workover fluids, gravel packing fluids, and any combination thereof), and a combination thereof.

The minimum viscosity of the target composition (e.g., fracturing fluids, drilling fluids, completion fluids, workover fluids, gravel packing fluids, and any combination thereof) acceptable for carrying the proppant is, or is about, 100 cp, 150 cp, 180 cp, or 200 cp. In some embodiments, the viscosity of the target composition (e.g., a fracturing fluid) is more than 100 cp, more than 150 cp, more than 180 cp, or more than 200 cp. In some embodiments, the delayed release of enzyme from the formulated enzyme breaker disclosed herein allows the viscosity of the target fluid to be maintained above that minimum level for the delay period before reduction.

The temperature at which the formulated enzyme breakers disclosed herein can be used in hydrocarbon recovery process (e.g., treatment of subterranean formation, break of fracturing fluids) can vary. It can be advantageous, in some embodiments, for the formulated enzyme breaker to exhibit significant enzymatic activity or maximal enzymatic activity in about the well temperature. In some embodiments, the formulated enzyme breaker is used in hydrocarbon recovery under a temperature that is, or is about, 70° F., 80° F., 90° F., 100° F., 110° F., 120° F., 130° F., 140° F., 150° F., 160° F., 170° F., 180° F., 190° F., 200° F., 210° F., 220° F., 230° F., 240° F., 250° F., 260° F., 270° F., 280° F., 290° F., 300° F., or a range between any two of these values. In some embodiments, the formulated enzyme breaker is used in hydrocarbon recovery under a temperature between 70° F. to 300° F., or 140° F. to 220° F.

Various fluids and substances used and/or produced in the hydrocarbon recovery process can be treated with the particles for well treatment and/or formulated enzyme breakers disclosed herein. Non-limiting examples of the fluids include fracturing fluids, drilling fluids, completion fluids, workover fluids, gravel packing fluids, and any combination thereof. In some embodiments, the fluid comprises one or more hydratable polymers. In some embodiments, the fluid comprises water, brine, alcohol, or any mixture thereof.

The pH of the fluids can also vary. For example, the pH of the fluid can be, or be about, 5.0 to 12.0. In some embodiments, the pH of the fluid is, or is about, 5.0, 5.5, 6.0, 6.5, 7.0, 7.5, 8.0, 8.5, 9.0, 9.5, 10.0, 10.5, 11.0, 11.5, 12.0, 12.5, 13.0, or a range between any two of these values. In some embodiments, the pH of the fluid is about 6.0 or higher, or about 6.5 or higher. In some embodiments, the pH of the fluid is about 7.0 or higher, or about 7.5 or higher. In some embodiments, the pH of the fluid is about 8.0 or higher, or about 8.5 or higher. In some embodiments, the pH of the fluid is greater than or equal to 9.0, or greater than or equal to about 9.5.

### Well Treatment Composition and Methods of Use Thereof

Also provided are well treatment compositions that comprise particles of well treatment disclosed herein. The well treatment composition can comprise any particles for well treatment disclosed herein, and any combinations thereof. In some embodiments, the well treatment composition comprises a plurality of the particles, one or more viscosifiers and one or more solvents. The composition can further comprise, for example, one or more cross-linking agents.

The well treatment composition can comprise, for example, any subterranean treatment fluid (e.g., fracturing fluid, drilling fluid, gravel packing fluid, completion fluid, or workover fluid) or any combination of the subterranean treatment fluids. In some embodiments, the well treatment composition comprises a viscosified fluid. In some embodiments, the well treatment composition is a fluid form.

Various viscosifiers can be present in the well treatment composition (e.g., well treatment fluid). For example, the viscosifier can comprise guar, substituted guar, cellulose, derivatized cellulose, xanthan, starch, polysaccharide, gelatin, polymers, synthetic polymer, or any combination thereof. Non-limiting examples of the substituted guar include hydroxylethyl guar, hydroxypropyl guar, carboxymethylhydroxyethyl guar, carboxymethylhydroxypropyl guar (CMHPG), and any combination thereof. Non-limiting examples of the derivatized cellulose include carboxymethyl cellulose, polyanoinic cellulose, hydroxyethyl cellulose, and any combination thereof.

Various solvents can be present in the well treatment composition (e.g., well treatment fluid). The solvent can be, for example, aqueous or organic-based. In some embodiments, the solvent is water (for example, fresh water, sea water, produced water, water from aquifers, or any combination thereof), brine, water with water-soluble organic compounds, or any combination thereof.

In some embodiments, the well treatment composition comprises a gelling agent (viscosifier). Non-limiting examples of the gelling agent or viscosifier include hydroxyethylcellulose, carboxymethyl cellulose, hydroxyalkyl guar, hydroxyalkyl cellulose, carboxyalkylhydroxy guar, carboxyalkylhydroxyalkyl guar, starch, gelatin, poly(vinyl alcohol), poly(ethylene imine), guar gum, xanthan gum, polysaccharide, cellulose, synthetic polymers, any derivatives thereof, and any combinations thereof. In some embodiments, the gelling agent or viscosifier is present in the well treatment composition in a concentration from about 15 pounds per thousand gallons (pptg) to about 80 pptg.

In some embodiments, the well treatment composition comprises one or more hydratable polymers. The hydratable polymers can be underivatized guars, derivatized guars, or any combination thereof. It can be advantageous in some embodiments to use underivatized guar. Examples of derivatized guars include, but are not limited to, hydroxypropyl guar and carboxymethyl hydroxypropyl guar.

It may advantageous, in some embodiments, to have the fluids used in hydrocarbon recovery process, for example, fracturing fluids, gravel packing fluids, completion fluids, workover fluids, and drilling fluids, to stay below a threshold pH value after being broken by the enzyme breaker to avoid reheal of the fluid (e.g., a cross-linked well treatment fluid) which will increase the viscosity of the broken fluid. As used herein, reheal of the fluid refers to, for example, re-gel or re-cross-link of the fluid. The threshold pH value can be, or be about, in some embodiments, 9.5, 9.45, 9.4, 9.35, 9.3, 9.25, 9.2, 9.15, 9.1, 9.05, 9.0, 8.95, 8.9, or a range between any two of these values. In some embodiments, the threshold pH value is 9.5. The particles for well treatment and/or the well treatment compositions disclosed herein can be configured, in some embodiments, to reduce the pH of a cross-linked well treatment fluid (e.g., a fracturing fluid, a gravel packing fluid, a completion fluid, a workover fluid, a drilling fluid, or any combination thereof) below the threshold pH where the cross-linked well treatment fluid can reheal. In some embodiments, the particle or the well treatment composition is configured to reduce the pH of a cross-linked well treatment fluid to below about 9.5. In some embodiments, for a delayed and complete break, it is advantageous that the ending pH of a cross-linked well treatment fluid is below 9.5 (after treatment with the particles or the well treatment composition disclosed herein) and that the enzyme selected is active in at least the range of pH 9.5. Unlike cellulases which are active only when the pH is reduced to neutral, the cellulase enzymes described herein (e.g., the polypeptides of SEQ ID NO: 2, SEQ ID NO:6, SEQ ID NO:9, SEQ ID NO:11, SEQ ID NO:13, SEQ ID NO:15, SEQ ID NO:17, SEQ ID NO:19, or SEQ ID NO:21 and the polypeptides encoded by SEQ ID NO: 1, SEQ ID NO: 3, SEQ ID NO:4, SEQ ID NO:5, SEQ ID NO:7, SEQ ID NO:8, SEQ ID NO:10, SEQ ID NO:12, SEQ ID NO:14, SEQ ID NO:16, SEQ ID NO:18, or SEQ ID NO:20), are active in the range of pH 9.5. This activity, in combination with a reduction in pH to below 9.5 (with the co-encapsulated acidifying agent, as described herein), provide a complete break of a cross-linked well treatment fluid.

Various cross-linking agents can be present in the well treatment composition and the fluids used in hydrocarbon recovery (e.g., fracturing fluids). In some embodiments, the crosslinking agent comprises boron derivatives, potassium periodate, potassium iodate, ferric iron derivatives, magnesium derivatives, and any combination thereof. Examples of crosslinking agents include, but are not limited to, borate ion, zirconate ion, titanate ion, and any combination thereof. In some embodiments, the cross-linking agent(s) is present in the well treatment composition in a concentration from about 0.5 gallons per thousand gallons (gpt) to about 5 gpt.

In some embodiments, the cross-linking agent comprises metal ions. For example, the cross-linking agent can comprises aluminum-, antimony-, zirconium-, and titanium-containing compounds, borates, boron releasing compounds, and any combination thereof. In some embodiments, the cross-linking agent comprises organotitanates. In some embodiments, the crosslinking agent is a material which supplies borate ions. Non-limiting examples of borate cross-linkers include organoborates, monoborates, polyborates, mineral borates, boric acid, sodium borate, including anhydrous or any hydrate, borate ores (e.g., colemanite or ulexite), and any other borate complexed to organic compounds to delay the release of the borate ion. It can be advantageous, in some embodiments, to use borate crosslinking agents as the cross-linking agent.

The well treatment composition disclosed herein can also, in some embodiments, comprise a plurality of proppant particulates. Particulates suitable for use may comprise any material suitable for use in subterranean operations. Suitable materials for these particulates include, but are not limited to, sand, bauxite, ceramic materials, glass materials, polymer materials, polytetrafluoroethylene materials, nut shell pieces, cured resinous particulates comprising nut shell pieces, seed shell pieces, cured resinous particulates comprising seed shell pieces, fruit pit pieces, cured resinous particulates comprising fruit pit pieces, wood, composite particulates, and any combinations thereof. The well treatment composition may also comprise a binder and a filler material wherein suitable filler materials include silica, alumina, fumed carbon, carbon black, graphite, mica, titanium dioxide, meta-silicate, calcium silicate, kaolin, talc, zirconia, boron, fly ash, hollow glass microspheres, solid glass, or any combinations thereof. The mean particulate size generally may range from about 2 mesh to about 400 mesh on the U.S. Sieve Series; however, in certain circumstances, other mean particulate sizes may be desired and will be entirely suitable for practice of the methods and compositions disclosed herein. In some embodiments, it can be advantageous to use particulates with mean particulates size distribution ranges of 6-12 mesh, 8-16 mesh, 12-20 mesh, 16-30 mesh, 20-40 mesh, 30-50 mesh, 40-60 mesh, 40-70 mesh, or 50-70 mesh on the U.S. Sieve Series. It should be understood that the term “particulate,” as used in this disclosure, includes all known shapes of materials, including substantially spherical materials, fibrous materials, polygonal materials (such as cubic materials), and any combinations thereof. Moreover, fibrous materials, that may or may not be used to bear the pressure of a closed fracture, may also be present in the compositions disclosed herein. In some embodiments, the particulates may be present in the well treatment composition (e.g., a well treatment fluid) in an amount in the range of from about 60 g/L or 0.5 pounds per gallon (“ppg”) to about 3500 g/L or 30 ppg by volume of the well treatment composition.

Any proppants conventionally known in the art can be used, including but are not limited to, quartz sand grains, glass beads, aluminum pellets, ceramics, plastic beads, including polyamides, and ultra-lightweight (ULW) particulates such as ground or crushed shells of nuts like walnut, coconut, pecan, almond, ivory nut, brazil nut, etc.; ground and crushed seed shells (including fruit pits) of seeds of fruits such as plum, olive, peach, cherry, apricot, etc.; ground and crushed seed shells of other plants such as maize (e.g., corn cobs or corn kernels), etc.; processed wood materials such as those derived from woods such as oak, hickory, walnut, poplar, mahogany, etc., including such woods that have been processed by grinding, chipping, or other form of particalization, processing, etc.

The well treatment compositions can be used to treat subterranean formations. In some embodiments, the method of treating a subterranean formation comprises: contacting the subterranean formation with a well treatment fluid, wherein the well treatment fluid comprises any of the particles for well treatment disclosed herein, one or more viscosifiers, and one or more solvents; and allowing the enzyme in the particles for well treatment to reduce the viscosity of the well treatment fluid.

The extent by which the enzyme can reduce the viscosity of the well treatment fluid can vary, and can be determined according to the specific use/purpose of the users. For example, the viscosity of the well treatment fluid after the enzyme treatment can be, or be about, 0.001%, 0.01%, 0.1%, 1%, 5%, 10%, 15%, 20%, 25%, 30%, 35%, 40%, 45%, 50%, 75%, or a range between any two of these values, of the viscosity of the well treatment fluid prior to enzyme treatment. In some embodiments, the enzyme reduces the viscosity of the well treatment fluid by about, or at least about, 10%, 20%, 30%, 40%, 50%, 60%, 65%, 75%, 80%, 85%, 90%, 95%, 100%, two folds, three folds, five folds, one order of magnitude, two orders of magnitude, three orders of magnitude, four orders of magnitude, five orders of magnitude, six orders of magnitude, seven orders of magnitude, or a range between any two of these values. In some embodiments, the enzyme completely breaks the well treatment fluid. In some embodiments, the enzyme breaks the well treatment fluid to a free flowing, water-thin fluid. In some embodiments, the enzyme reduces the viscosity of well treatment fluid to a viscosity of less than 10 cP as measured with VISCOlab 4000 from Cambridge Viscosity.

In some embodiments, other than the acidifier present in the particles for well treatment, the well treatment fluid does not comprise any additional pH reducing agent. In some embodiments, besides the acidifier present in the particles for well treatment, the well treatment fluid comprises one or more additional pH reducing agents. In some embodiments, the method for treating a subterranean formation comprises contacting an additional pH reducing agent with the well treatment fluid to adjust the pH value of the well treatment fluid. In some embodiments, the enzyme completely breaks the well treatment fluid in the absence of any additional pH reducing agent. The pH reducing agent can be, for example, any of the acidifying agents disclosed herein.

In some embodiments, the method for treating a subterranean formation comprises: providing a viscosified treatment fluid having a first viscosity, wherein the viscosified treatment fluid comprises: a gelling agent, a proppant, an aqueous-base fluid, and any of the formulated enzyme breakers disclosed herein; introducing the viscosified treatment fluid into the subterranean formation; creating or enhancing a fracture in the subterranean formation; and allowing the formulated enzyme breaker to release the acidifier and enzyme so as to reduce the viscosity of the viscosified treatment fluid to a second viscosity.

As would be appreciated by one of ordinary skill in the art, the time at which the formulated enzyme breaker comprising the particles for well treatment is to be admixed with a viscosified treatment fluid can vary. In some embodiments, the formulated enzyme breaker is admixed with the viscosified treatment fluid prior to introduction into the subterranean formation. In some embodiments, the formulated enzyme breaker is admixed simultaneously into the viscosified treatment fluid while the viscosifier is being introduced into the subterranean formation. In some embodiments, the formulated enzyme breaker is admixed into the viscosified treatment fluid after the viscosifier has been introduced into the subterranean formation. In some embodiments, the viscosified treatment fluid additionally comprises a cross-linking agent. The viscosified fluid can be an aqueous-based or organic-solvent-based fluid. The aqueous-base fluid can be, for example, any fluid that is water-based. Examples of aqueous-based fluid include, but are not limited to, salt water, brine, water, and the like.

In some embodiments, the well treatment fluid can also comprise unencapsulated breakers. Examples of unencapsulated breakers include, but are not limited to, oxidizers such as ammonium persulfate, sodium persulfate, sodium chlorite, magnesium peroxide, magnesium oxide, enzymes, and any combination thereof. Such mixtures of encapsulated and unencapsulated breakers can, in some embodiments, speed up the breaking process when desirable. In some embodiments, the well treatment fluid can comprise more than one type of encapsulated breakers. For example, the well treatment fluid can comprise the formulated enzyme breakers disclosed herein and one or more additional encapsulated breakers. Examples of the additional encapsulated breakers include, but are not limited to, oxidizers (e.g., ammonium persulfate, sodium persulfate, sodium chlorite, magnesium peroxide, magnesium oxide, and any combination thereof), enzymes, and any combination thereof. The formulation and method for encapsulating other breakers can be the same, similar or different from the enzyme encapsulation described herein.

In some embodiments, the method for treating a subterranean formation comprises: providing a viscosified treatment fluid having a first viscosity, wherein the viscosified treatment fluid comprises a crosslinked gelling agent formed by a reaction comprising a gelling agent and a crosslinking agent, a proppant, an aqueous-base fluid, and an formulated enzyme breaker comprising any of the particles disclosed herein for well treatment; introducing the viscosified treatment fluid into the subterranean formation; creating or enhancing a fracture in the subterranean formation; and allowing the formulated enzyme breaker to expose the enzyme breaker to the viscosified treatment fluid over time so as to reduce the viscosity of the viscosified treatment fluid. In some embodiments, the viscosified treatment fluid is brought to a complete break after the enzyme treatment.

In some embodiments, the formulated enzyme breaker is added to the viscosified treatment fluid after the gelling agent and crosslinking agent have crosslinked.

The formulated enzyme breakers disclosed herein are capable of breaking fluid substrate (e.g., fracturing fluid) at various temperatures, for example about 80° F. to about 250° F. In some embodiments, the fluid substrate is brought to a complete break by the formulated enzyme breaker. In some embodiments, the formulated enzyme breaker breaks the fluid substrate at a temperature that is, or is about, 80° F., 90° F., 100° F., 110° F., 120° F., 130° F., 140° F., 150° F., 160° F., 170° F., 180° F., 190° F., 200° F., 210° F., 220° F., 230° F., 240° F., 250° F., a range between any two of these values.

The formulated enzyme breakers disclosed herein are capable of breaking fluid substrate (e.g., fracturing fluid) at various pH level, for example about pH 5 to about pH 11. In some embodiments, the formulated enzyme breaker can bring the fluid substrate to a complete break. In some embodiments, the formulated enzyme breaker breaks the fluid substrate at a pH that is, or is about, 5, 5.5, 6, 6.5, 7, 7.5, 8, 8.5, 8.6, 8.7, 8.8, 8.9, 9, 9.1, 9.2, 9.3, 9.4, 9.5, 9.6, 9.7, 9.8, 9.9, 10, 10.5, 11, or a range between any two of these values. In some embodiments, the formulated enzyme breaker is capable of bringing the fluid substrate to a complete break in the absence of any additional pH reducing agent or composition (e.g. an ester, or acidic buffer). In some embodiments, the formulated enzyme breaker delays break for a period of about 20 minutes to about 240 minutes at a temperature of about 80° F. to 250° F., and a pH in a range of 6-11. In some embodiments, the formulated enzyme breaker delays break for a period of about 20 minutes to about 240 minutes at a temperature of 80° F.-250° F., and a pH in a range of 6-11, in the absence of any additional pH reducing agent or composition.

In some embodiments, the enzyme is cellulase. In some embodiments, the enzyme is mannanase. The cellulase or mannanase may hydrolyze the guar polymer at temperatures in excess of 160° F. as well as in excess of 180° F. In fact, the cellulase may hydrolyze the guar polymer at temperatures in excess of 185° F. and even in excess of 195° F. In addition, the cellulase or mannanase may be used in combination with other enzymes and/or oxidative breakers to degrade guar gels over broader temperature and pH ranges.

### EXAMPLES

Additional embodiments are disclosed in further detail in the following examples, which are not in any way intended to limit the scope of the claims.

### Example 1

### Enzyme and Various Acidifiers

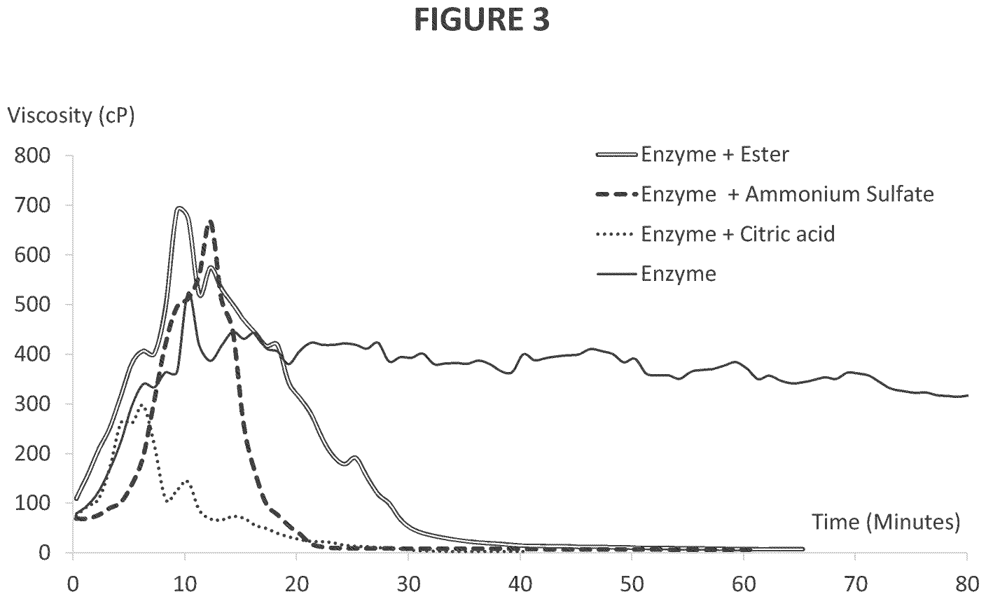

Rheology studies were performed using guar solutions with the addition of various acidifiers (ethyl acetoacetate ester, ammonium sulfate, citric acid) and the cellulase enzyme embodied by SEQ ID NO: 2 in liquid form. A control assay was performed with guar solution and the enzyme embodied by SEQ ID NO: 2 only. Guar gum at 25 pptg was hydrated in water for 45 min by vigorous stirring, followed by the addition of surfactant, clay stabilizer, and pH adjuster to reach pH=10.5. Upon addition of delayed cross-linker, the enzyme and acidifiers were added at following final concentrations: a) 6 mU/mL Enzyme+5 mM Ester (ethyl acetoacetate); b) 6 mU/mL Enzyme+2.5 mM ammonium sulfate; c) 6 mU/mL Enzyme+2.5 mM citric acid; d) (Control) 25 mU/mL Enzyme, with no other additions. Viscosity was measured using low pressure viscometer, at 167° F., with ˜15 min temperature ramping. Complete break of guar was examined by pouring test at ambient temperature (broken guar displayed viscosity <6 cP). The results of the studies are as displayed in graph in FIG. 3. All solutions reached complete break aside from the control.

### Example 2

### Acidifiers Reverse Guar Cross-Linking but do not Break Guar to a Complete Break

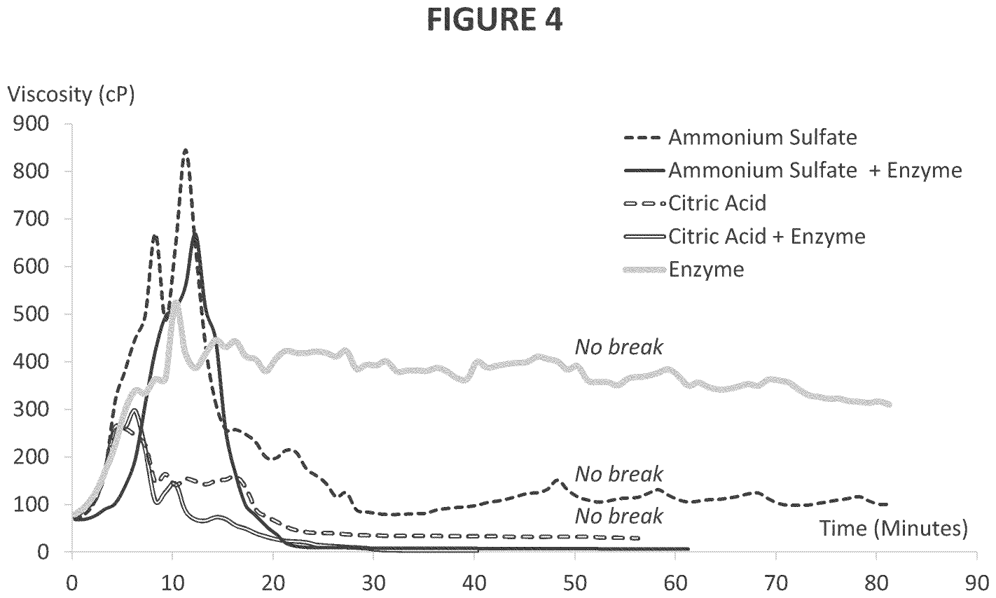

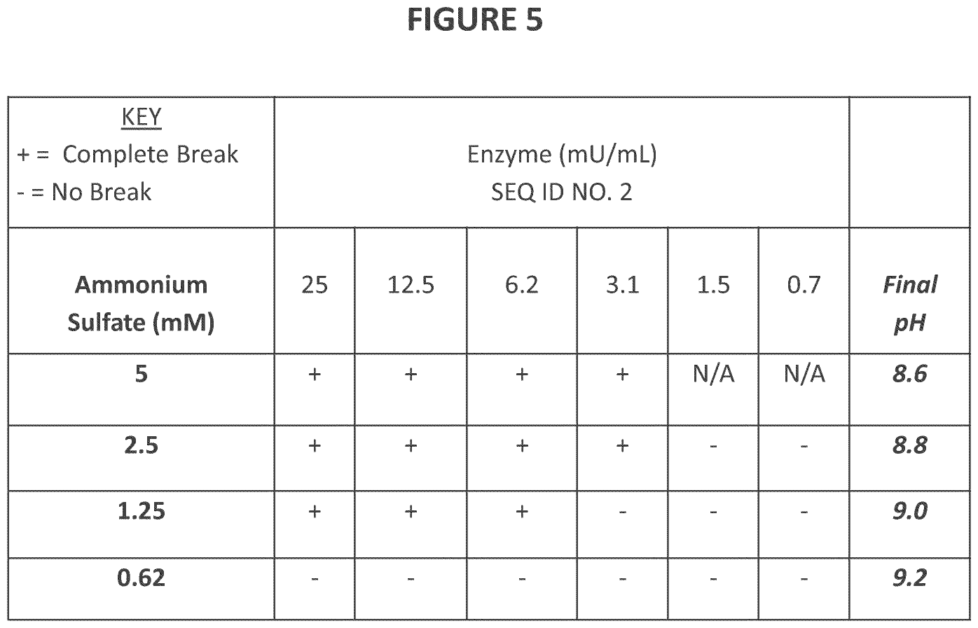

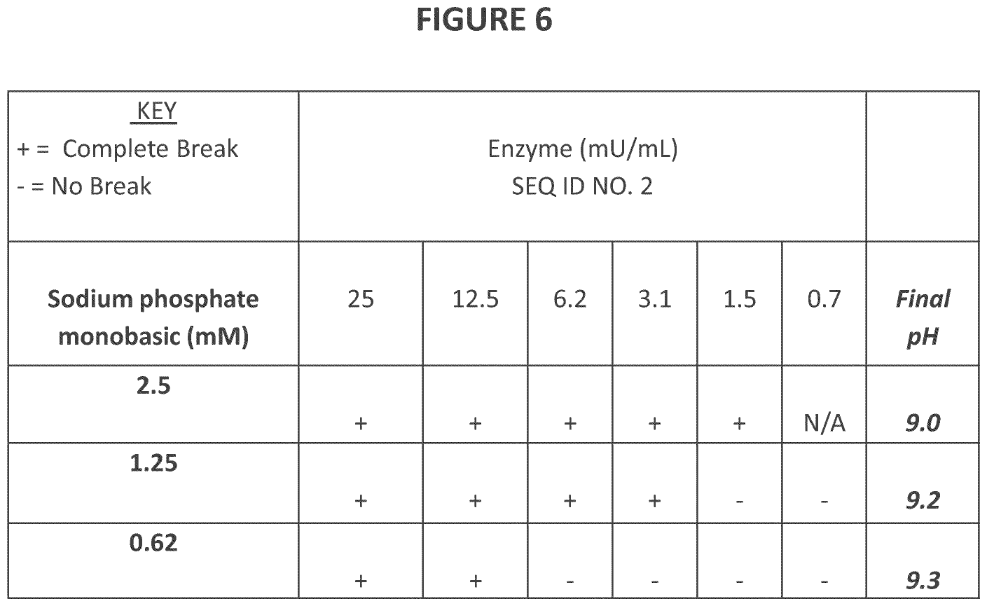

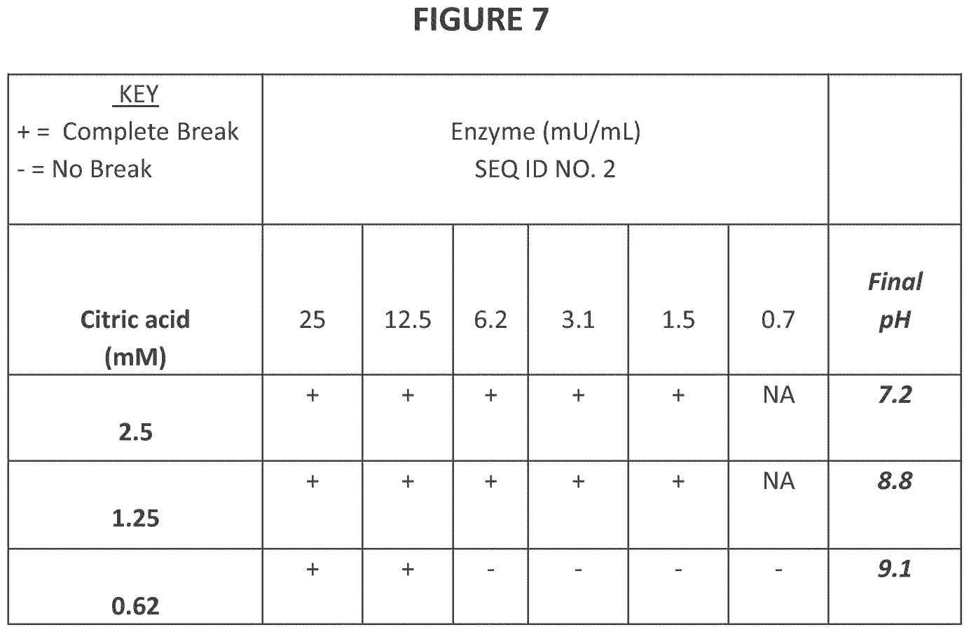

To test the influence of acidifiers (without enzyme) upon guar rheology, rheology studies were performed in the absence of enzyme as described in Example 1, and at 2.5 mM final concentration (broken lines). The positive control (solid lines) was guar treated with both acidifier (2.5 mM) and enzyme (6 mU/mL). The negative control (gray line) was guar treatment with enzyme alone (25 mU/mL). Although acidifiers decreased guar viscosity, the guar in these samples did not reach a complete break in the absence of enzyme. The results are shown in FIG. 4. Acidifiers shifted the equilibrium from cross-linked guar to linear guar (non-cross-linked) due to a change in the environmental pH (from initial pH˜10.5 to final pH˜7.2-9, see FIG. 5-7).

### Example 3

### Acidifier Levels Required for Complete Break

In order to calculate the optimal concentrations of enzyme and acidifiers necessary to completely break guar, studies like in Example 1 were carried out using enzyme concentrations of 0.7-25 mU/mL and acidifier concentrations between 0.67 mM-5 mM. Acidifiers used for this example were Ammonium Sulfate, Sodium Phosphate Monobasic, and Citric Acid. Complete break of guar was examined by pouring test at ambient temperature (broken guar displayed viscosity <6 cP). Final pH was recorded to establish the impact of the acidifier on the guar pH. Results are displayed in FIGS. 5, 6, and 7. The optimal concentrations were used to design the ratios between enzyme and the acidifier used as a carrier in the encapsulated sample.

### Example 4

### Without Ester Addition, Effects of Acidifier Carrier Particle

Enzyme was attached to an acidifier carrier, as well as to a non-acidifying carrier, to test the ability of enzyme to break the guar in the absence of extrinsic ester addition.
(a) Enzyme affixed to non-acidifying carrier: Microcrystalline cellulose spheres were coated with a 0.75% solution of 131,000 ave. MW polyvinyl alcohol (PVA130) containing 200 U/mL of enzyme in a Wurster bowl configuration for a total weight gain of 0.375%. These loaded carriers were then further coated with 1.5% PVA 130 for a weight gain of 0.75% and a final activity level of 100 U/g. Samples were dried at 40° C. (104° F.).
(b) Enzyme affixed to acidifying carrier: Granular ammonium sulfate was coated in a Wurster bowl configuration with a 1.5% sodium alginate solution containing 320 U/mL enzyme for a total weight gain of 0.5625%. This loaded carrier was then further coated with a 1.5% sodium alginate solution containing 20% maltodextrin and 10% Kaolin for a total weight gain of 11.8% and a final activity of 106.7 U/g. Samples were dried at 40° C. (104° F.).

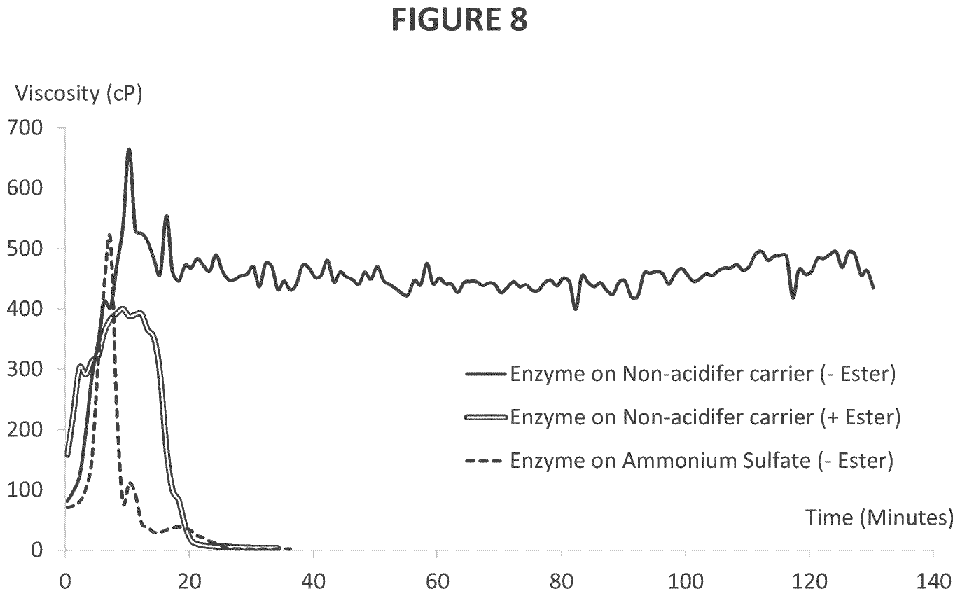

Dried samples were added to the cross-linked guar solution to reach final concentration of 25 mU/mL in the absence or presence of ester, ethyl acetoacetate, at 5 mM. The experiment was carried out in the same conditions as described in Example 1. Results are shown in FIG. 8. Both the enzyme affixed to acidifier carrier sample and the enzyme affixed to non-acidifier carrier plus ester sample produced full break of guar solution, while the sample with enzyme affixed to non-acidifier carrier without ester did not break the guar.

### Example 5

### Carrier and Encapsulation

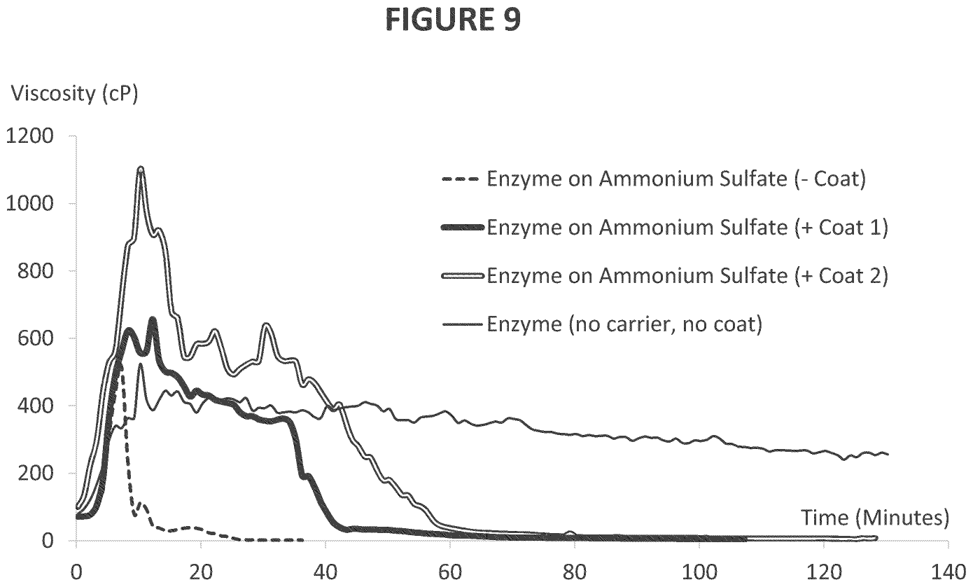

Dried sample containing enzyme affixed to ammonium sulfate carrier (no coat) was further coated with a protective coat followed by an impermeable compatible polymeric compound. Samples were tested in the low pressure viscometer at 25 mU/mL final enzyme concentration, as described in example 1. Results are displayed in FIG. 9.
(a) Coat 1: Granular ammonium sulfate was coated in a Wurster bowl configuration with a 1.5% sodium alginate solution containing 320 U/mL enzyme for a total weight gain of 0.5625%. The loaded carrier was then further coated with a 1.5% sodium alginate solution containing 20% maltodextrin and 10% Kaolin for a total weight gain of 11.8% over the previous combination. This material was then loaded into a coating pan and coated with a 55% polyvinylidene chloride dispersion for a 25% weight gain and a final activity of 86 U/g.
(b) Coat 2: Granular ammonium sulfate was coated in a Wurster bowl configuration with a 1.5% sodium alginate solution containing 300 U/mL enzyme for a total weight gain of 0.6%. This loaded carrier was then further coated with a 1% sodium alginate solution containing 20% maltodextrin and 10% Kaolin for a total weight gain of 15.5%. This material was then loaded into a coating pan and coated with a 55% polyvinylidene chloride dispersion for a 50% weight gain and a final activity of 52 U/g.

As shown in FIG. 9, addition of a polymeric coat onto the enzyme affixed to ammonium sulfate carrier generated a delay in enzyme release from the particles and, consequently, in guar break, a feature which is essential for effective fracturing.

### Example 6

### Carrier and Encapsulation with Two Successive Coats

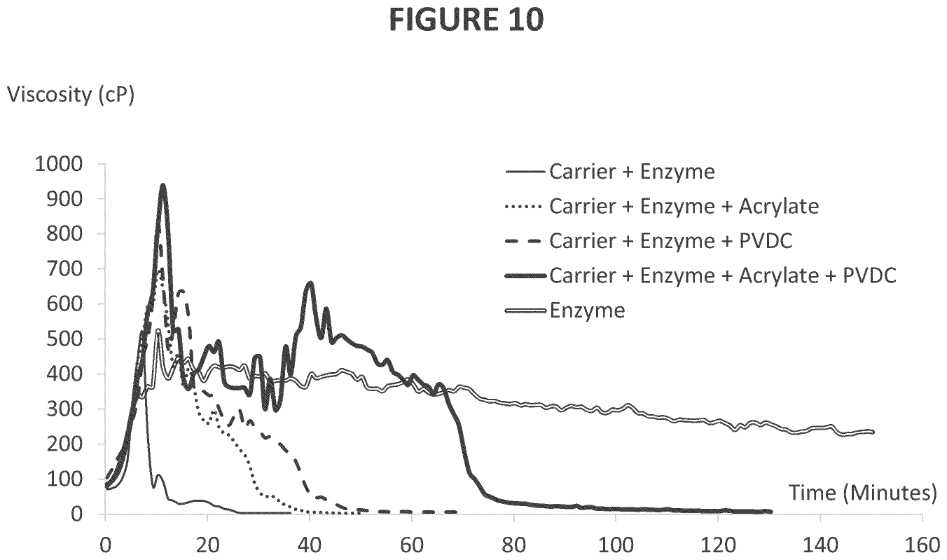

Dried sample containing enzyme affixed to ammonium sulfate carrier was coated with two successive layers of polymeric encapsulant material to improve the delay release. Samples were tested in the low pressure viscometer at 25 mU/mL final enzyme concentration, as described in example 1. Results are shown in FIG. 10.
(a) Carrier plus enzyme: A 20 to 60 mesh fraction of granulated ammonium sulfate was Wurster coated with a 1% sodium alginate solution containing 67 U/mL of enzyme for a weight gain of 0.5% and final activity of 50 U/mL.
(b) Carrier plus enzyme plus acrylate: A 20 to 60 mesh fraction of granulated ammonium sulfate was Wurster coated with a 1% sodium alginate solution containing 67 U/mL of enzyme for a weight gain of 0.5%. The loaded carrier was then further coated by placing the loaded carrier into a coating pan and sprayed with a 55% dispersion of acrylic polymer for a weight gain of 50% and a final activity of 35 U/g.
(c) Carrier plus enzyme plus PVDC: A 20 to 60 mesh fraction of granulated ammonium sulfate was Wurster coated with a 1% sodium alginate solution containing 67 U/mL of enzyme for a weight gain of 0.5%. The loaded carrier was then placed into a coating pan and sprayed with a 55% dispersion of polyvinylidene chloride for a weight gain of 50% and a final activity of 35 U/g.
(d) Carrier plus enzyme plus acrylate plus PVDC: a 20 to 60 mesh fraction of granulated ammonium sulfate was Wurster coated with a 1% sodium alginate solution containing 67 U/mL of enzyme for a weight gain of 0.5%. The loaded carrier was then further coated by placing the loaded carrier into a coating pan and sprayed with a 55% dispersion of acrylic polymer for a weight gain from the original batch size of 50% followed by a 55% dispersion of polyvinylidene chloride for an additional weight gain from the original batch size of 40% and a final activity of 25 U/g.

FIG. 10 shows that encapsulating the enzyme in two successive layers of polymers (acrylate followed PVDC) is beneficial to increasing the delayed activity of the enzyme and, consequently, breaking the guar.

### Example 7

### Heat Tolerance of Granulated Acidifier Carrier with Enzyme

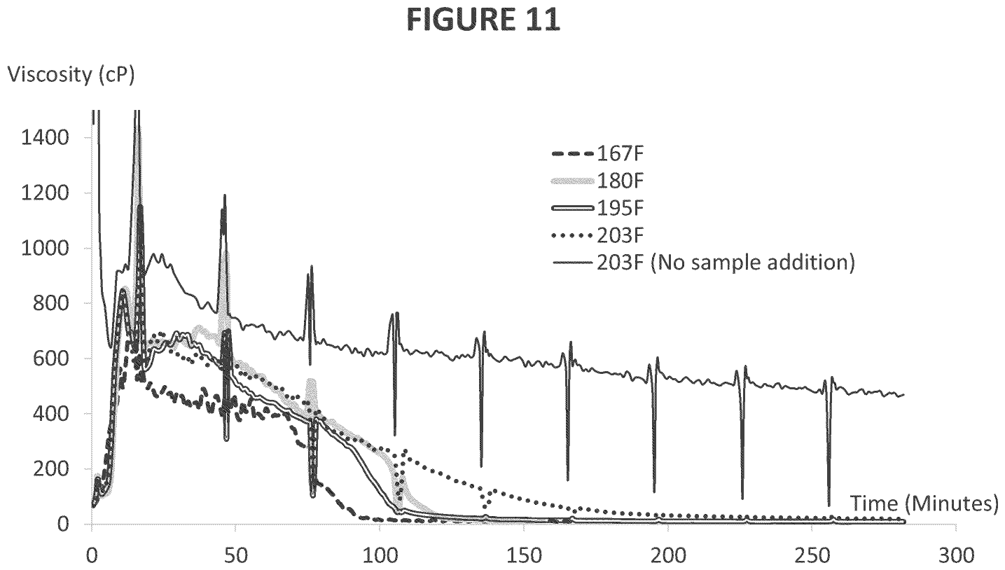

Dried granules containing enzyme affixed to acidifier, and stabilizers were coated with adequate layers of polymeric encapsulant as described above to ensure enzyme survival at high temperatures. Samples were tested in high pressure viscometer at 167°, 180°, 195°, 203° F. temperature points and 500 psi final pressure at various dosages ensuring complete guar break (70 mU/mL-170 mU/mL). Results are displayed in FIG. 11. Granulation was achieved in a high shear mixer with equal portions of microcrystalline cellulose, mannitol, and ammonium sulfate using polyvinyl pyrrolidone as a binder which was directly mixed with the enzyme solution. The loaded carrier was then dried in an oven and passed through a 12 mesh screen with a 60 mesh screen being used to remove any fine particles. The sized, dried granules of enzyme affixed to acidifier carrier were then placed in a coating pan and spray coated with acrylic dispersion for total weight gain of 100% from the original material.

As can be seen in FIG. 11, encapsulated enzyme affixed to acidifier reached a complete break at each temperature point (167°, 180°, 195°, 203° F.); while the sample lacking the encapsulated enzyme did not break guar at 203° F.

### Example 8

### Heat Tolerance of Encapsulated Acidifiers with Enzyme

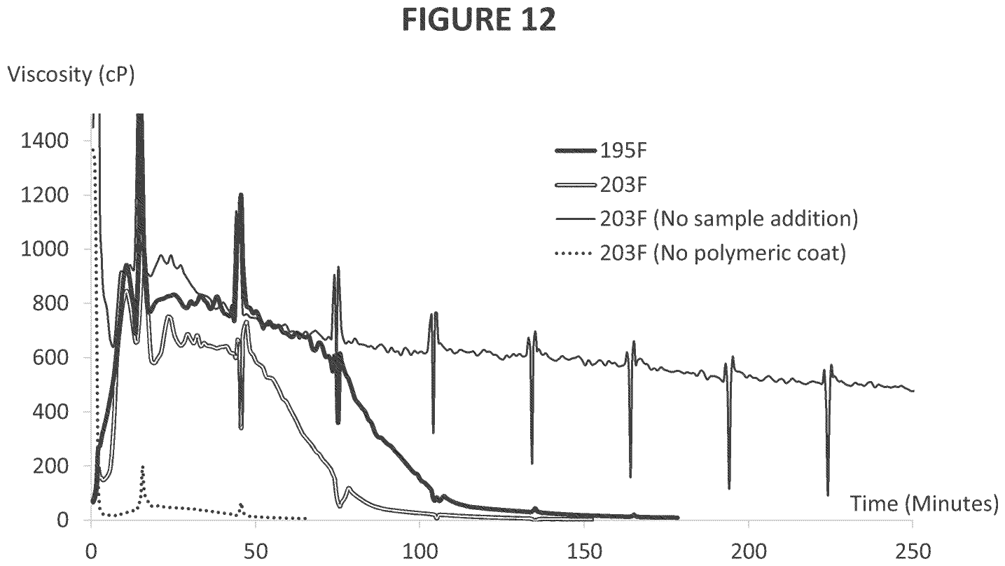

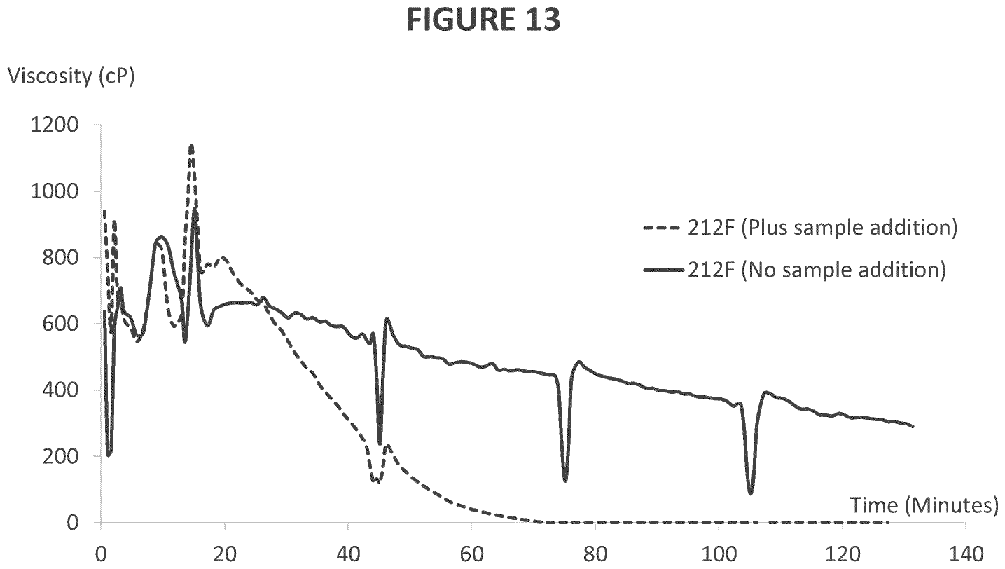

Dried samples of enzyme affixed to acidifier carrier were coated with thick layers of polymeric encapsulant material to ensure enzyme survival at high temperatures. Ammonium sulfate was placed in a Wurster coater and sprayed with an enzyme solution containing 1% sodium alginate. These cores were then placed into a coating pan and sprayed with acrylic dispersion for a weight gain of 200%. Samples were tested in high pressure viscometer at 195° F.−212° F. and 500 psi final pressure at dosages ensuring complete guar break (80 mU/mL-125 mU/mL). Results are displayed in FIG. 12 and FIG. 13.

### Example 9

### Shelf Life and Storage Stability of Encapsulated Acidifiers with Enzyme

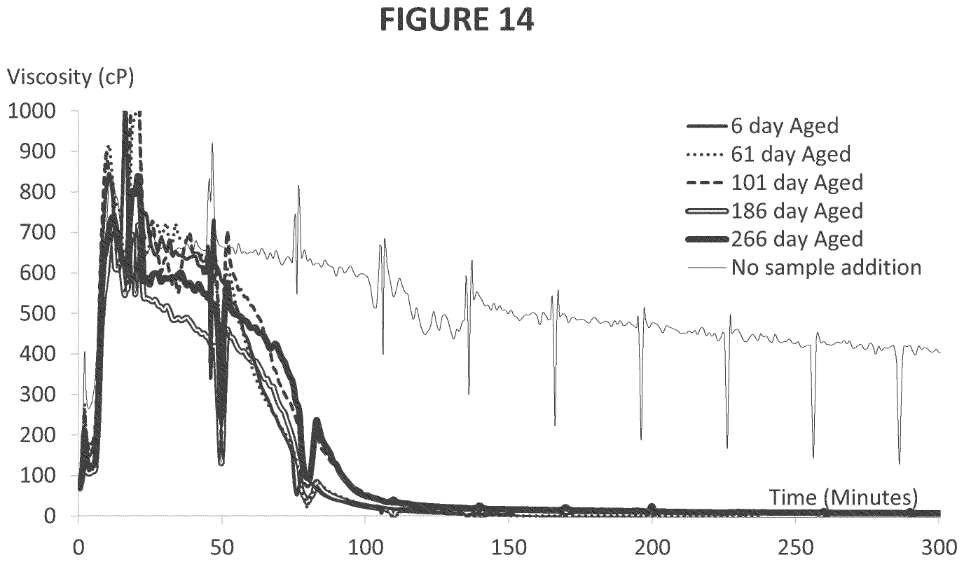

Dried samples of enzyme affixed to acidifier carrier were coated with thick layers of polymeric encapsulant material to ensure enzyme survival at high temperatures. Ammonium sulfate was placed in a Wurster coater and sprayed with an enzyme solution containing 1% sodium alginate. These cores were then placed into a coating pan and sprayed with acrylic dispersion for a weight gain of 200%. Samples were left to age in plastic bottles kept at room temperature (15-25° C.) for the shown amount of time. Samples were then tested in high pressure viscometer at pH10.5, 203° F. and 500 psi final pressure at dosages ensuring complete guar break (˜100 mU/mL or 13 pptg). Results are shown in FIG. 14, which shows that consistent break profile was achieved from samples stored for different length of times.

### Example 10

### Dose Dependence of Encapsulated Acidifiers with Enzyme

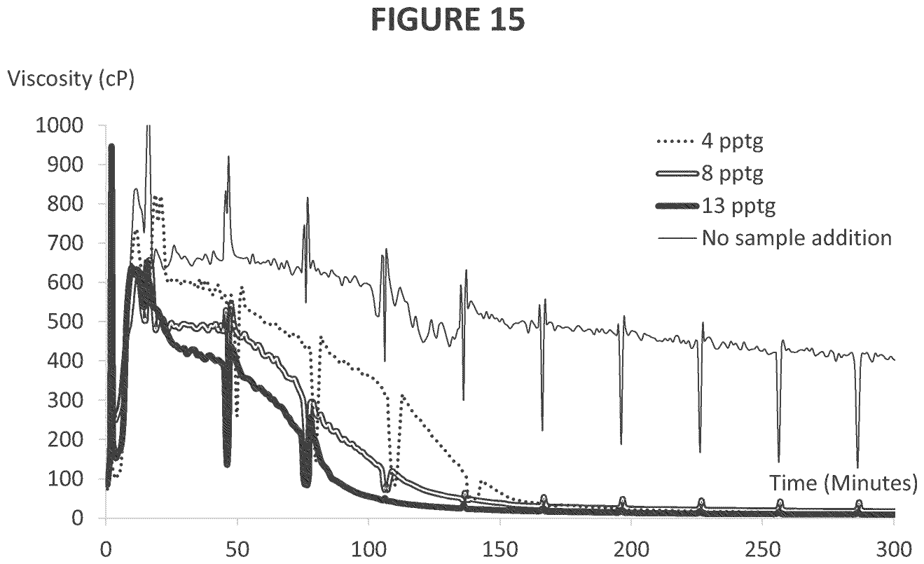

Dried samples of enzyme affixed to acidifier carrier were coated with two layers of polymeric encapsulant material. Ammonium sulfate was placed in a Wurster coater and sprayed with an enzyme solution containing 5% poly(vinylpyrrolidone). These cores were then placed into a coating pan and sprayed with a dispersion of styrene/n-butyl acrylate copolymer for a weight gain of 80%, followed by a second spraying with a hard film forming acrylic emulsion. Samples were tested in high pressure viscometer at pH10.5, 203° F. and 500 psi final pressure at the described dosages (4.0-13.0 pptg encapsulated samples or 66-214 mU/mL of final enzyme concentration in cross-linked guar fluid). Results are displayed in FIG. 15.

### Example 11

### Effect of Fluid Ending pH on Obtaining a Complete Break of Cross-Linked Guar Fluids

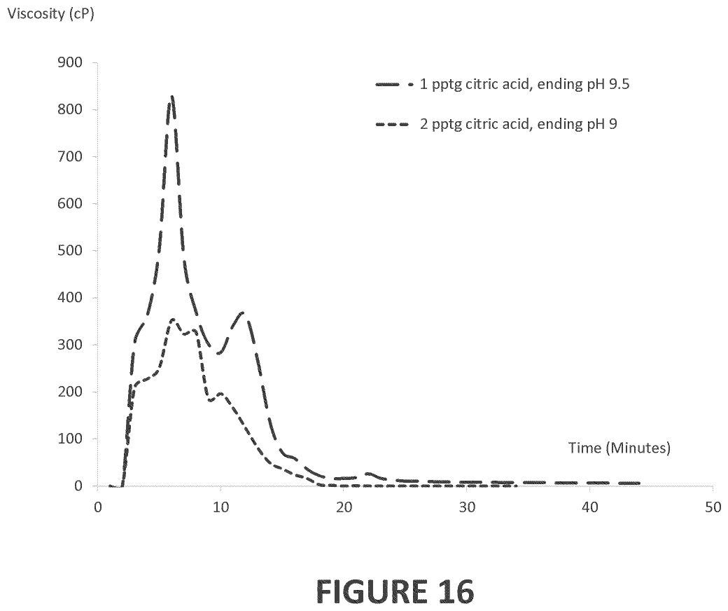

Non-encapsulated cellulase having the amino acid sequence of SEQ ID NO:2 at 32 mU/mL with 1 pptg citric acid or 2 pptg citric acid were applied to cross-linked guar fluid at 25 pptg at pH 10.5. Rheology tests were performed with Grace Viscometer with temperature of 165° F. and 500 psi. The results are shown in FIG. 16.

As shown in FIG. 16, guar gum fluid with ending pH 9.0 (below 9.5) was completely broken (with viscosity of 0 cP) at ambient temperature. Although the guar gum fluid with ending pH 9.5 exhibited 0 cP viscosity at 165° F., the cross-linked gel rehealed with a viscosity of >200 cP at ambient temperature.

### Example 12

### Effect of Fluid Ending pH on Obtaining a Complete Break of Cross-Linked Guar Fluids

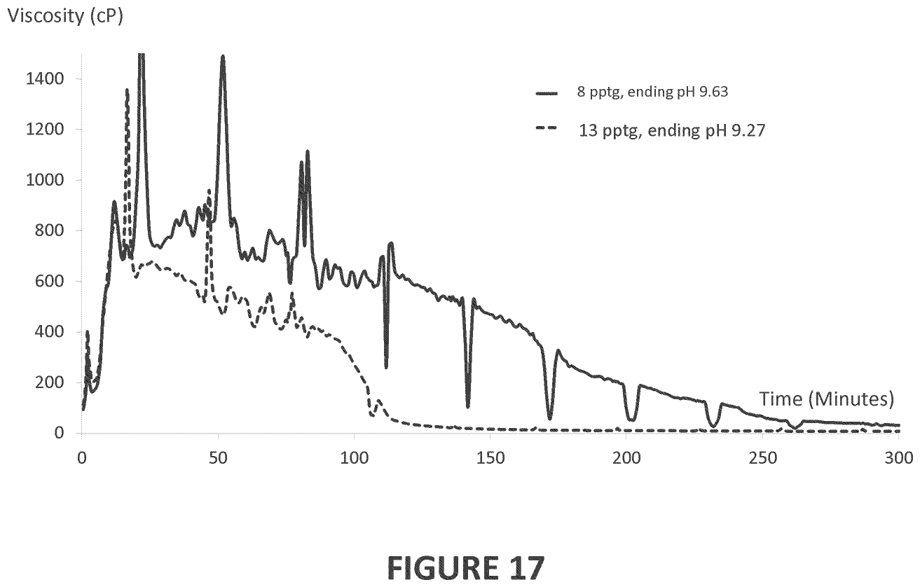

Dried samples of enzyme affixed to an acidifying carrier were coated with a thick layer of polymeric encapsulant material to ensure enzyme survival at high temperatures. To produce the enzyme and acidifier core, ammonium sulfate was placed in a Wurster coater and sprayed with an enzyme (cellulase having the amino acid sequence of SEQ ID NO:2) solution containing 2.5% poly(vinylpyrrolidone). These cores were then placed into a coating pan and sprayed with an acrylic dispersion for a weight gain of 200%. Rheology testing of the resulting encapsulated particles was conducted in a high pressure viscometer at 203° F. and 500 psi final pressure at dosages ensuring complete guar break (167 mU/mL-278 mU/mL). The cross-linked fluid system had an initial pH of 11. The encapsulated particles were added to the cross-linked fluid at 8 pptg and 13 pptg. The results are shown in FIG. 17.

As shown in FIG. 17, the fluid with the ending pH of 9.27 exhibited a complete break. In contrast, the fluid with the ending pH of 9.63 was not completely broken, with the fluid becoming rehealed at ambient temperature.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A well treatment composition comprising:
a plurality of particles, wherein the particle comprises an enzyme-containing core having an acidifying agent and an enzyme; and a shell configured to at least partially encapsulate the enzyme-containing core;
a viscosifier; and
a solvent.

2. The well treatment composition of claim 1, wherein the composition further comprises a cross-linking agent.

3. The well treatment composition of claim 1, wherein the composition is configured to reduce the pH of a cross-linked well treatment fluid below a threshold pH value at and above which the fluid can reheal.

4. The well treatment composition of claim 3, wherein the well treatment fluid is a fracturing fluid, a gravel packing fluid, a completion fluid, a workover fluid, a drilling fluid, or any combination thereof.

5. The well treatment composition of claim 3, wherein the threshold pH value is 9.5.

6. The well treatment composition of claim 1, wherein the acidifying agent is granular ammonium sulfate; and the shell at least partially encapsulating the enzyme-containing core allows controlled release of the enzyme from the particle.

7. The well treatment composition of claim 1, wherein the enzyme is present on the outer surface of the enzyme-containing core; or is dispersed within the enzyme-containing core; or is dispersed within the enzyme containing core and present on the outer surface of the enzyme containing core.

8. The well treatment composition of claim 1, wherein the enzyme containing core additionally comprises a binding agent, a carrier, and/or a stabilizer, wherein:
(a) the binding agent is selected from polyvinylpyrrolidone, polyvinyl alcohol, alginate, polyethylene glycol, wax, xanthan gum, polyvinyl acetate, carrageenans, starch, maltodextrin, hydroxypropyl cellulose, hydroxypropyl methylcellulose, methylcellulose, carboxymethyl cellulose, styrene acrylic dispersions, or any combination thereof;
(b) the carrier is selected from fibrous and microcrystalline cellulose, sodium sulfate, sodium chloride, monocalcium phosphate, dicalcium phosphate, tricalcium phosphate, monosodium phosphate, disodium phosphate, trisodium phosphate, monopotassium phosphate, dipotassium phosphate, tripotassium phosphate, calcium carbonate, diatomaceous earth, zeolite, starch, or any combination thereof; and
(c) the stabilizer is selected from mannitol, trehalose, sorbitol, xylitol, sucrose, microcrystalline cellulose, starch, sodium chloride, sodium sulfate, ammonium sulfate, or any combination thereof.

9. The well treatment composition of claim 1, wherein the shell comprises a polymer, a homopolymer, a copolymer, or any combination thereof; wherein the polymer comprises one or more of the monomers selected from the group consisting of methacrylic acid, methacrylic ester, methacrylic amide, methacrylic nitril, acrylic acid, acrylic ester, acrylic amide, acrylic nitril, and vinyl monomers selected from styrene and alpha methyl styrene; and

[symbol]

10. The well treatment composition of claim 1, wherein the shell comprises ethylcellulose, acrylic resin, plastics, methacrylate, acrylate, acrylic acetate, polyvinylidene chloride (PVDC), nitrocellulose, polyurethane, wax, polyethylene, polyethylene glycol, polyvinylalcohol, polyester, polylactic acid, polyglycolic acid, copolymers of polylactic and polyglycolic acids, polyvinyl acetate, vinyl acetate acrylic copolymer, alginates, agar, styrene-acrylate copolymer, styrene/n-butyl acrylic copolymer, or any combination thereof.

11. The well treatment composition of claim 1, wherein at least one of the enzymes is a cellulase, a hemicellulase, a pectinase, a xanthanase, a mannanase, a galactosidase, or an amylase, and wherein the enzyme is a thermostable or thermotolerant enzyme.

12. The well treatment composition of claim 1, wherein the particle comprises one or more additional coatings outside of or underneath the shell, and wherein at least one of the additional coatings is a polymeric protective coating or a polymeric polishing coating.

13. The well treatment composition of claim 1, wherein the size of the particle is about 7 mesh to about 60 mesh on the U.S. Sieve Series.

14. The well treatment composition of claim 1, wherein the shell substantially encapsulates the enzyme containing core.

15. The well treatment composition of claim 1, wherein the viscosifier comprises guar, substituted guar, cellulose, derivatized cellulose, xanthan, starch, polysaccharide, gelatin, polymer, synthetic polymer, or any combination thereof, and wherein the substituted guar is hydroxylethyl guar, hydroxypropyl guar, carboxymethylhydroxyethyl guar, carboxymethylhydroxypropyl guar (CMHPG), or the derivatized cellulose is carboxymethyl cellulose, polyanoinic cellulose, hydroxyethyl cellulose, or any combination thereof.

16. The well treatment composition of claim 1, wherein the solvent is aqueous or organic-based.

17. The well treatment composition of claim 16, the solvent is fresh water, sea water, brine, produced water, water from aquifers, water with water-soluble organic compounds, or any mixture thereof.

18. A method of treating a subterranean formation, comprising
contacting the subterranean formation with the well treatment fluid of claim 4; and
allowing the enzyme to reduce the viscosity of the well treatment fluid.

19. The method of claim 18, wherein the well treatment fluid is a fracturing fluid, a gravel packing fluid, a completion fluid, a workover fluid, or a drilling fluid, or any combination thereof.

20. A method for making particles for well treatment, comprising
contacting an enzyme with a solid acidifying agent to form an enzyme-containing core; and
encapsulating the enzyme-containing core with one or more shells to form the particles for well treatment, wherein each of the shells is configured to at least partially encapsulate the enzyme-containing core.

21. The method of claim 20, wherein the contacting step comprises attaching the enzyme to the solid acidifying agent by a non-perforated pan coating process, a pan coating process, a fluidized bed coating process, a spray drying process, or any combination thereof; or wherein the contacting step comprises spraying a solution comprising the enzyme onto the solid acidifying agent.

22. A method for making particles for well treatment, comprising
mixing an enzyme and a solid acidifying agent to form a mixture;
granulating the mixture to form an enzyme-containing core; and
encapsulating the enzyme-containing core with one or more shells to form the particles for well treatment, wherein each of the shells is configured to at least partially encapsulate the enzyme-containing core.

23. The method of claim 22, further comprising drying the enzyme-containing core before encapsulating the enzyme-containing core with the shells.

24. The method of claim 22, wherein the mixture further comprises a binder, a stabilizer, an inert carrier, or any combination thereof.

25. The method of claim 22, wherein granulating the mixture to form an enzyme-containing core is achieved by a wet granulation process.

26. The method of claim 25, wherein the wet granulation process comprises extrusion, centrifugal extrusion, spheronization, batch high shear granulation, continuous high shear mixing, disc granulation, drum granulation, spray drying, fluid bed agglomeration, fluid bed granulation and/or layering, prilling, or any combination thereof.

27. The method of claim 25, wherein the fluid bed granulation and/or layering comprises bottom spray, tangential spray, and spouted bed.

28. The method of claim 22, wherein the enzyme-containing core is encapsulated by a non-perforated pan coating process, a pan coating process, a fluidized bed coating process, a spray drying process, or any combination thereof.

29. The method of claim 22, wherein the fluidized bed coating process is a bottom spray process, a Wurster process, a top spray process, a tangential spray process, a spouted bed process, a modified fluidized bed coating process, or a continuous fluidized bed coating process, or any combination thereof.

30. The method of claim 22, wherein the shell comprises a polymer, a homopolymer, a copolymer, or any combination thereof, and wherein polymer comprises one or more of the monomers selected from the group consisting of methacrylic acid, methacrylic ester, methacrylic amide, methacrylic nitril, acrylic acid, acrylic ester, acrylic amide, acrylic nitril, and vinyl monomers.

31. The method of claim 22, wherein the shell comprises ethylcellulose, acrylic resin, plastics, methacrylate, acrylate, acrylic acetate, polyvinylidene chloride (PVDC), nitrocellulose, polyurethane, wax, polyethylene, polyethylene glycol, polyvinylalcohol, polyester, polylactic acid, polyglycolic acid, copolymers of polylactic and polyglycolic acids, polyvinyl acetate, vinyl acetate acrylic copolymer, alginates, agar, styrene-acrylate copolymer, styrene/n-butyl acrylic copolymer, or any combination thereof.

32. The method of claim 22, wherein the weight gain of solid content upon encapsulating the enzyme-containing core with the one or more shells is about 20% to about 250%.

33. The method of claim 22, wherein the encapsulating step comprising curing the particles at an elevated temperature to promote formation of at least one of the shells.

34. The method of claim 33, wherein the elevated temperature is between about 25° C. to about 80° C.

35. The method of claim 22, wherein the one or more shells are successive shells.

36. A particle for well treatment, comprising
an enzyme-containing core, wherein the enzyme-containing core comprises an acidifying agent and an enzyme; wherein the acidifying agent is in the form of solid particle, the acidifying agent serves as a carrier for the enzyme, and the acidifying agent is granular ammonium sulfate; and
a shell which at least partially encapsulates the enzyme-containing core, wherein the shell allows controlled release of the enzyme from the particle.
